(19) United States
(12) Patent Application Publication
(10) Pub. No.: US 2025/0073171 A1
(43) Pub. Date: Mar. 6, 2025

(54) DRUG DELIVERY SYSTEM FOR THE TREATMENT OF FEMALE SEXUAL INTEREST AND AROUSAL DISORDER

(71) Applicant: Freya Pharma Solutions Holding B.V., Amsterdam (NL)

(72) Inventor: Marius Jan van der Mooren, Beuningen (NL)

(21) Appl. No.: 18/826,043

(22) Filed: Sep. 5, 2024

Related U.S. Application Data
(60) Provisional application No. 63/580,671, filed on Sep. 5, 2023.

(30) Foreign Application Priority Data
Sep. 5, 2023 (EP) 23195531.1

(51) Int. Cl. A61K 9/20; A61K 9/24; A61K 9/28; A61K 31/519; A61K 31/568

(52) U.S. Cl. CPC A61K 9/2054; A61K 9/209; A61K 9/2846; A61K 31/519; A61K 31/568

(57) ABSTRACT

This disclosure relates to a dual release drug delivery system for use in enhancing female sexual desire in the treatment of Female Sexual Interest and Arousal Disorder (FSIAD). the composition comprises a core comprising cellulose, a filler selected from an organic and/or an inorganic salt, and a first active ingredient comprising a PDE 5 inhibitor for delayed immediate release. a first coating surrounding the core, comprising a hydrophobic polymer and a hydrophilic substance; and a second coating surrounding the first coating, comprising a second active ingredient which is a testosterone, or a functional analogue or derivative of testosterone, for immediate release. The delayed release of the first active ingredient is an immediate release occurring between 2 to 6 hours after the release of the second active ingredient; and the second active ingredient is present in an amount equivalent to from 0.3 to 1.5 mg of testosterone, or in an amount equivalent to no less than 1.0 mg of testosterone. In addition, the disclosure also relates to a method for enhancing female sexual desire in the treatment of Female Sexual Interest and Arousal Disorder (FSIAD).

This application claims the benefit of the filing date of U.S. Provisional Patent Application Ser. No. 63/580,671, filed Sep. 5, 2023, for “DRUG DELIVERY SYSTEM FOR THE TREATMENT OF FEMALE SEXUAL INTEREST AND AROUSAL DISORDER,” and European Patent Application No. EP23195531.1, filed Sep. 5, 2023, the disclosure of each of which is hereby incorporated herein in its entirety by this reference.

### TECHNICAL FIELD

The application relates to the field of drug formulation and drug delivery, particularly for the diagnosis and treatment of female sexual interest/arousal disorder (FSIAD). It specifically relates to the influence of the combination of testosterone or an analogue thereof and a PDE5 inhibitor on sexual desire (as well as relevant related outcomes such as, for example, sexual/bodily arousal and distress) in female subjects with FSIAD. The disclosure further relates to methods for enhancing female sexual desire in the treatment of FSIAD, an algorithm useful for determining dosage of such drug formulations and delivery systems for specific patient groups, and methods for determining a suitable (starting) dosage and subsequently optimizing the dosage, if and when necessary.

### BACKGROUND

Sexual interest refers to the motivation to engage in sexual activity. It is also commonly referred to as “desire,” “sex drive,” and “sexual appetite,” and describes the sexual feelings motivating a person to seek some type of sexual activity, whether partnered or alone. Sexual arousal is conceptualized as the second phase of the sexual response cycle and defined by both physical and mental readiness for sexual activity. Physiological changes (for example erection in males, vaginal swelling and lubrication in females) occur in the body to prepare for a sexual interaction.

Low sexual desire is the most common sexual complaint in women. As a result, many women suffer from sexual dissatisfaction and related distress which often negatively interferes with their quality of life. Female Sexual Interest/Arousal Disorder (FSIAD) refers to the lack of, or significantly reduced, sexual interest/arousal in women. Typical symptoms of FSIAD may include: absent or reduced interest in sexual activity; absent or reduced sexual thoughts or fantasies; no or reduced initiation of sexual activity, and typically unreceptive to a partner's attempts to initiate; absent or reduced sexual excitement or pleasure in almost all or all sexual encounters; absent or reduced sexual interest/arousal in response to any internal or external sexual cues; and absent or reduced genital or non-genital sensations during sexual activity in all or almost all sexual encounters. Typically in patients with FSIAD, these symptoms have caused clinically significant distress and have persisted for a minimum of six months. The disorder is specified by severity level and subtyped into lifelong versus acquired, generalized versus situational. Previous studies have examined the prevalence of low sexual interest and low sexual arousal in women. One of the most frequently cited prevalence study found low sexual interest in 22% of women in the general U.S. population (Laumann, Paik, Rosen, & Page, 1999). In a survey of women from 29 countries, rates of self-reported low sexual interest ranged from 26 to 43% (Laumann et al., 2005). Taking levels of distress into account, rates may range from 7.3% (Bancroft, Loftus, & Long, 2003) to 23% (Witting et al., 2008), depending on a woman's age, cultural background, and reproductive status.

The FDA's new diagnosis guidelines for FSIAD are merged from two previously separate diagnoses (Hypoactive Sexual Desire Disorder (HSDD) and Female Sexual Arousal Disorder. (FSAD)). Treatment of FSIAD may include psychotherapy and/or medication. Currently, limited drug treatments are available globally to treat women with FSIAD. When assessing the efficacy of various treatments of FSIAD, women's sexuality, particularly desire, often requires careful consideration of the multifactorial nature of it, and single-item scales, such as SSEs (satisfying sexual events) are often too simplistic and fail to account for the subtle multidimensional subjective issue that contribute to the construct of sexual desire.

It would therefore be highly desirable to provide a drug delivery system for the treatment for FSIAD, that is safe and effective, with a focus on effectively enhancing patients' sexual desire. It is also desirable to provide practical guidance on the determination of (starting) dosages of such a drug delivery system (pharmaceutical composition) for a patient, and additional pharmaceutical compositions that cater for patients who may have special needs.

### BRIEF SUMMARY

Provided are new drug delivery systems and methods of treatment that are effective for enhancing sexual desire in the treatment of FSIAD, in particular acquired FSIAD, and that do not have serious side effects. In the context of the disclosure, enhancing sexual desire encompasses enhancing an individual's sexual interests, thoughts and/or excitements, reducing sexual anxieties and/or distress, encouraging sexual and bodily arousal, as well other physical and psychological activities that prepare the individual, bodily and/or mentally, for sexual activities. The drug delivery system of the disclosure is a dual route/dual release fixed dose system of testosterone, for example sublingual testosterone, and a PDE5 inhibitor, for example an oral PDE5 inhibitor, with the aim to synergistically enhance both central and peripheral processes. The inventors of the disclosure also found that there are a number of factors in the general female population that may influence the efficacy of a combination treatment of FSIAD with testosterone (an immediate release formulation) and a PDE5 inhibitor (in a delayed immediate release formulation). These factors comprise the menopausal status of a woman, the use of combined oral contraceptives (COCs) or combined hormone replacement therapy (HRT), the sex hormone binding globulin (SHBG) levels, the albumin levels and the (body mass index) (BMI). Often these factors will be inter-related. For an all-comer population the 0.5 mg, 50 mg dual dosage of testosterone and sildenafil respectively will be efficacious, but for instance for post-menopausal women, in particular those with very low BMI a higher testosterone dosage (up to 4 mg) may be useful. Providing safe and efficacious dosages of both testosterone and sildenafil (or equivalents of either) for women that may not be served as well with the all comer dosage are an important aspect of the disclosure.

In one aspect, the disclosure provides a dual release drug delivery system (pharmaceutical composition) for use in enhancing female sexual desire in the treatment of Female Sexual Interest and Arousal Disorder (FSIAD). The composition comprises a core, a first coating surrounding the core, and a second coating surrounding the first coating. The core comprises cellulose, a filler selected from an organic and/or an inorganic salt, and a first active ingredient for delayed immediate release. The first coating comprises hydrophobic polymer and a hydrophilic substance. And the second coating comprises a second active ingredient for immediate release. The first active ingredient comprises a PDE5 inhibitor, and the second active ingredient is a testosterone, or a functional analogue or derivative of testosterone. In the dual drug delivery system of the disclosure, the delayed release of the first active ingredient is an immediate release occurred between 2 to 6 hours after the release of the second active ingredient; and the second active ingredient is present in an amount equivalent to from 0.3 to 1.5 mg of testosterone, or no less than 1.0 mg, for example 1-5 mg, preferably 1-4 mg, for example 2.0 mg, 2.5 mg, 3.0 mg, 3.5 mg, 4.0 mg testosterone. The disclosure is particularly useful in the treatment of acquired FSIAD, but can also be used for the treatment of other subtypes of FSIAD.

Treatments with a dosage of testosterone combined with a PDE5 inhibitor produce alterations in brain and bodily functions which will make learning of positive associations between sexual stimuli, genital arousal and subjective experience possible, thereby enhancing female sexual desire.

The term “first active ingredient” refers to the ingredient that is present in the core. The first ingredient may be a single active ingredient or a mixture of two or more active ingredients. A first active ingredient that is present in the core of a drug delivery system according to the disclosure is one which is released after a defined period of time.

According to this part of the disclosure, the first active ingredient comprises a PDE5-inhibitor, such as vardenafil, sildenafil or tadalafil or any of the other known PDE5-inhibitors, or a combination thereof. Further non-limiting examples of PDE5 inhibitors are: E-4021, E-8010, E-4010, AWD-12-217 (zaprinast), AWD 12-210, UK-343,664, UK-369003, UK-357903, BMS-341400, BMS-223131, FR226807, FR-229934, EMR-6203, Sch-51866, IC485, TA-1790 (avanafil), DA-8159 (udenafil), NCX-911 or KS-505a. Other examples can be found in WO 96/26940.

A preferred PDE5-inhibitor according to the disclosure is sildenafil which is preferably administered as sildenafil citrate (1-[[3-(6,7-dihydro-1-methyl-7-oxo-3-propyl-1Hpyrazolo[4,3-cr|pyrimidin-5-yl)-4-ethoxyphenyl]sulfonyl]-4-methylpiperazine citrate).

Preferably, the first active ingredient is present in the drug delivery system of the disclosure in an amount of at least 10 mg, at least 25 mg, at least 40 mg, at least 50 mg, at least 60 mg, at least 70 mg, at least 75 mg, at least 80 mg, at least 90 mg, at least 100 mg.

In some (non-limiting) embodiments, the PDE5 inhibitor may be present in an amount even higher than 100 mg, for example higher than 110 mg or 120 mg. Preferably, the PDE5 inhibitor is present in the pharmaceutical composition of the disclosure in an amount of no more than 200 mg, no more than 190 mg, no more than 180 mg, no more than 170 mg, no more than 160 mg, no more than 150 mg. Preferably, the PDE5 inhibitor is present in the pharmaceutical composition of the disclosure in an amount of 10 to 150 mg, 20 to 140 mg, 30 to 130 mg, 40 to 120 mg, 50 to 110 mg or 50 to 100 mg. In some preferred embodiment of the disclosure, the PDE5 inhibitor is present in the pharmaceutical composition of the disclosure in an amount of 50 mg, or 75 mg, or 100 mg, or 125 mg, or 150 mg.

In some preferred embodiments of the disclosure, the first active ingredient is sildenafil (or a salt thereof, for example sildenafil citrate). It may present in the drug delivery system of the disclosure in an amount of from 10 to 120 mg. Preferably, sildenafil is present in the drug delivery system of the disclosure in an amount of from 20 to 100 mg, or from 20 to 75 mg. For example, the first active ingredient is sildenafil (or a salt thereof, for example sildenafil citrate) present in an amount of 25 mg or 50 mg or 75 mg. In an alternative embodiment, the first active ingredient is sildenafil (or a salt thereof, for example, sildenafil citrate) present in an amount of 100 mg. In a preferred embodiment of the disclosure, the drug delivery system comprises testosterone in an amount of 1.0 mg and sildenafil (or a salt thereof, for example sildenafil citrate) present in an amount of 100 mg. In alternative but still preferred embodiments of the disclosure, the drug delivery system comprises testosterone in an amount of 1.0 mg and sildenafil (or a salt thereof, for example sildenafil citrate) present in an amount of 25 mg or 50 mg or 75 mg.

The first active ingredient is present in the core in a relative amount of preferably between 0.1 and 60% (w/w; based on the total weight of the core), more preferred between 0.1 and 30% (w/w; based on the total weight of the core), more preferred between 5 and 25% (w/w based on the total weight of the core).

Optionally, the cellulose is present in the core in a relative amount of preferably between 10 and 60% (w/w based on the total weight of the core), more preferred between 10 and 50% (w/w based on the total weight of the core (w/w based on the total weight of the core). The term cellulose comprises powdered cellulose, agglomerated cellulose, microcrystalline cellulose and/or combinations thereof. The term cellulose includes purified cellulose, methylcellulose, hydroxypropyl methylcellulose, and carboxy methyl cellulose. Powdered cellulose is composed mainly of cellulose obtained by decomposing pulp. Microcrystalline cellulose comprises a special grade of alpha cellulose.

A preferred cellulose is microcrystalline cellulose. A preferred microcrystalline cellulose has a nominal particle size of between 30 and 250 m, preferably of between 50 and 180 m. A further preferred microcrystalline cellulose comprises a moisture of between 0.1 and 7.5%, more preferred between 1 and 5.0%. A preferred microcrystalline cellulose is selected from microcrystalline cellulose with a nominal particle size of 50 μm and a moisture of 3.0 to 5.0% such as, for example, Avicel PH 101; a microcrystalline cellulose with a nominal particle size of 100 μm and a moisture of 3.0 to 5.0% such as, for example, Avicel PH 102; and a microcrystalline cellulose with a nominal particle size of 180 m and a moisture less than 1.5% such as, for example, Avicel PH 200. The amount of the microcrystalline cellulose is preferably more than 10% (w/w; based on the total weight of the core), more preferred more than 20% (w/w), more preferred more than 30%, most preferred more than about 35%. It is further preferred that the amount of microcrystalline cellulose is less than 60%, more preferred less than 50%, more preferred less than 45% (w/w, based on the total weight of the core).

Optionally, the filler is present in the core in a relative amount of preferably between 10 and 70% (w/w based on the total weight of the core), more preferred in an amount of between 10 and 60% (w/w based on the total weight of the core). more preferred between 20% and 60% (w/w), more preferred between 30% and 50% (w/w), such as, for example, 35% (w/w).

The filler is selected from the group of an organic salt and an inorganic salt. An organic salt is preferably selected from calcium citrate, magnesium citrate, calcium lactate, sodium lactate, magnesium lactate, calcium fumarate and magnesium fumarate. A most preferred filler is an inorganic salt. An inorganic salt according to the disclosure is preferably selected from calcium sulphate dehydrate, calcium silicate, silicium phosphate, calcium carbonate, anhydrous dibasic calcium phosphate, dibasic calcium phosphate monohydrate, tribasic calcium phosphate, sodium phosphate, sodium chloride, potassium phosphate, potassium sulphate, potassium chloride, sodium carbonate, magnesium carbonate, and magnesium oxide. The total amount of a soluble filler such as sodium lactate and sodium chloride is preferably below 50% (w/w; based on the total weight of the core). The selection of a filler is further determined by the intrinsic stability of the active ingredient in the core in combination with a filler or combination of fillers, as is known to the person skilled in the art.

The core may further comprise a lubricant such as magnesium stearate, talc and the like. A preferred core comprises anhydrous dibasic calcium phosphate and magnesium stearate. The amount of the anhydrous dibasic calcium phosphate is preferably more than 10% (w/w; based on the total weight of the core), more preferred more than 20% (w/w), more preferred more than 30%, most preferred more than about 35%. It is further preferred that the amount of anhydrous dibasic calcium phosphate is less than 70%, more preferred less than 60%, more preferred less than 50%, more preferred less than 45% (w/w, based on the total weight of the core). The amount of magnesium stearate is preferably between 0.1% (w/w; based on the total weight of the core) and 10% (w/w), more preferred between 0.5 and 5% (w/w). The core additionally may comprise one or more disintegrants that, as a pure material, form a gel upon exposure to an aqueous liquid. A preferred disintegrant comprises one of more of a water-insoluble, gel-forming disintegrant, such as cross-linked sodium carboxy methylcellulose, sodium starch glycolate and/or cross-linked polyvinylpyrrolidone. When present, the disintegrant such as a water-insoluble, gel-forming disintegrant is preferably present in a relative amount of between 0.5 and 20% (w/w). Disintegrants are substances or a mixture of substances that facilitate the breakup or disintegration of a tablet. Break up of a tablet results in smaller particles of which the ingredients, including the first active ingredient, are more rapidly available for uptake, compared to a whole tablet. Drug dissolution can be improved significantly with the addition of disintegrating ingredients into the formulation. Preferred disintegrants induce disintegration of a tablet by wicking, deformation, and/or the induction of electric repulsive forces between particles. A preferred disintegrant according to the disclosure is selected from sodium starch glycolate (PRIMOJEL®), cross-linked sodium carboxymethyl cellulose, for example ACDISOL®, cross-linked polyvinylpyrrolidone (Crospovidone) and low-substituted hydroxypropyl cellulose (L-HPC) having a hydroxypropoxyl content in the range of 5.0 to 16.0% by weight and an apparent average degree of polymerization in the range of 350 to 700. The L-HPC preferably has a low particle size, preferably below 10 microns average particle size, more preferred below 5 micron, such as, for example, LH41. The water-insoluble, gel-forming disintegrant is preferably present in a relative amount of between 0.0 and 6% (w/w). The amount of the water-insoluble gel-forming disintegrant is preferably less than 6% (w/w; based on the total weight of the core), more preferred less than 5% (w/w), most preferred less than 4%.

A preferred composition of a core according to the disclosure comprises a first active ingredient, a microcrystalline cellulose, for example PHARMACEL® pH102 or PHARMACEL® pH200, anhydrous dicalcium phosphate, a crosslinked sodium carboxy methylcellulose, for example croscarmellose, and magnesium stearate. Microcrystalline cellulose and crosslinked sodium carboxy methylcellulose are preferably present in a ratio of between about 6:1 (w/w) to 14:1 (w/w), preferably between 7.5 (w/w) and 12.5 (w/w). Preferred ratios are about 10:1 (w/w) and about 8:1 (w/w). An effect of such ratio is that the core, while gel-forming, does not substantially swell prior to disintegration. A preferred ratio of anhydrous dibasic calcium phosphate and microcrystalline cellulose is between 3:1 (w/w) and 1:3 (w/w), more preferred between 2:1 (w/w) and 1:2 (w/w), most preferred in about 1:1 (w/w). The total weight of a core according to the disclosure is preferably between 50 and 500 milligram, more preferred between 200 and 400 milligram, more preferred between 300 and 400 milligram, such as about 340 milligram.

The core is preferably pressed or compacted into a solid. A preferred core is a tablet. The term “tablet” encompasses a “capsule” and a “caplet.” The preferred size of the core of a drug delivery system according to the disclosure ranges from a few millimeters to about one centimeter. The core may comprise further excipients including, but not limited to, diluents, binders or granulating ingredients, a carbohydrate such as starch, a starch derivative such as starch acetate and/or maltodextrin, a polyol such as xylitol, sorbitol and/or mannitol, lactose such as a-lactosemonohydrate, anhydrous a-lactose, anhydrous 6-lactose, spray-dried lactose, and/or agglomerated lactose, a sugar such as dextrose, maltose, dextrate and/or inulin, or combinations thereof, glidants (flow aids) and lubricants to ensure efficient tabletting, and sweeteners or flavors to enhance taste.

A core according to the disclosure is surrounded by at least one coating. The first coating comprises a hydrophobic polymer and a (water-soluble and/or water-insoluble) hydrophilic substance.

A hydrophobic coating polymer according to the disclosure is preferably selected from water-insoluble coating materials such as cellulose derivates and polymethacrylates that are generated, for example, by copolymerization of methacrylate monomers with hydrophobic groups. Preferred polymethacrylate hydrophobic polymers are EUDRAGIT® RL, EUDRAGIT® RS, EUDRAGIT® NE, and EUDRAGIT® S.

Preferred cellulose derivates are selected from ethylcellulose and derivatives thereof. In some preferred embodiments of the disclosure, the hydrophobic polymer is a hydrophobic polymeric ethylcellulose. A most preferred hydrophobic polymer of the first coating of a drug delivery system according to the disclosure comprises ethylcellulose. Ethylcellulose forms a mechanically weak hydrophobic film that ruptures easily. Preferably, the release of the active ingredient from the core is caused by rupture of the coating after a pre-defined lag-time. The core may, for example, contain a first ingredient in combination with a water-insoluble, gel-forming disintegrant that disintegrates upon contact with an aqueous medium. The formation of pores in the hydrophobic film, and the influx of water into the core, causes the rupture of the ethylcellulose coating. When the coating is ruptured, the core disintegrates within minutes followed by the release of the drug. A preferred ethylcellulose is ETHOCEL®.

The hydrophilic substance according to the disclosure preferably is a water-insoluble hydrophilic substance, preferably a water-insoluble hydrophilic polymer. It is further preferred that the first coating comprises pores prior to exposure to an aqueous liquid. The pores function as channels that interconnect the core with the outer surface of the inner coat for controlling the entry of aqueous liquid into the core. The pores are present, for example, when the water-insoluble hydrophilic substance is or comprises a water-insoluble hydrophilic polymer, preferably cellulose. Preferred celluloses are cellulose derivatives such as, for example, hydroxypropylcellulose, crosslinked hydroxyethylcellulose, crosslinked hydroxypropylmethylcellulose and microcrystalline cellulose. Cellulose formed channels that connect the drug-containing core with the outside of the tablet. The cellulose thereby controls the rate at which water is being transported through the channels into the core. When sufficient water reaches the core, the core loses its structural integrity. The core will disintegrate, followed by rupture of the coating and release of the drug. A preferred cellulose is a microcrystalline cellulose with a nominal particle size of between 20 and 200 micron and a moisture of less than 5%. A preferred microcrystalline cellulose comprises a microcrystalline cellulose with a nominal particle size of about 150 micron and a moisture of 3.0 to 5.0% such as, for example, AVICEL® PH-102 SCG; a microcrystalline cellulose with a nominal particle size of about 100 micron and a moisture less than 5.0% such as, for example AVICEL® HFE-102; a microcrystalline cellulose with a nominal particle size of about 20 micron and a moisture less than 5.0% such as, for example, AVICEL® PH-105. Further preferred water insoluble hydrophilic substances include dicalcium phosphate.

An advantage of using smaller particles of less than 50 micron, e.g., AVICEL® PH-105, is that the coating suspension has better flow properties, which improves the overall film coating process. A preferred first coating comprises ETHOCEL® and Avicel PH-105 as a water-insoluble hydrophylic substance. Preferred mass ratios of a hydrophobic coating polymer such as ETHOCEL® and a water-insoluble hydrophilic substance such as Avicel are between 1:5 and 5:1, more preferred between 1:4 and 3:1, more preferred between 1:3 and 2:1, most preferred about 1:2.

In another embodiment, a hydrophilic substance according to the disclosure preferably is a water-soluble hydrophilic substance. This coating preferably does not comprise pores or only a few pores prior to exposure to an aqueous liquid. It is preferred that the water-soluble hydrophilic substance forms pores in the hydrophobic polymer upon exposure to an aqueous liquid. A preferred water-soluble hydrophilic substance comprises lactose, mannitol and/or sodium chloride. A preferred lactose is PHARMATOSE®. A preferred first coating comprises ETHOCEL® and lactose as a water-soluble hydrophilic substance. Preferred mass ratios of a hydrophobic coating polymer such as ETHOCEL® and a water-soluble hydrophilic substance such as lactose are between 1:5 and 5:1, more preferred between 1:3 and 3:1, more preferred between 1:2 and 2:1, most preferred about 1:1.

The relative amount of a first coating is preferably between 4 and 20% (w/w; based on the total weight of the drug delivery system), more preferred between 8 and 15% (w/w), most preferred about 12% (w/w). Therefore, a preferred first coating has a weight of between 10 and 75 milligram, more preferred between 25 and 50 milligram, most preferred about 40 milligram.

Preferably, the first coating does not comprise a drug. The first coating may comprise a plasticizer such as, for example, dibutyl phthalate, triethyl citrate, acetyl triethyl citrate, dibutyl sebacate, diethyl phthalate, triacetin and/or tributyl citrate. When present, the plasticizer is preferably present in an amount of at most 0.5% (w/w; based on the total weight of the dual release drug delivery system). Preferably, the first coating does not comprise a plasticizer.

The first coating is preferably sprayed, for example with a nozzle, onto the core. For this, the hydrophobic polymer and water-soluble and/or water-insoluble hydrophilic substance are suspended or dissolved, for example in water or an organic solvent or a mixture thereof, and sprayed onto the core until a predetermined average thickness of the first coating is obtained. A preferred organic solvent is an alcohol, for example ethanol. The amount of the first coating is preferably between about 0.5 and 30% (w/w) of the total weight of the time controlled, immediate release drug delivery system, more preferred between about 1 and 20% (w/w).

A dual release drug delivery system according to the disclosure allows delayed release of a first active ingredient after hydration of the drug delivery system. The delayed release is an immediate release, and the timing is controlled in part by the thickness of the first coating, which is preferably sprayed onto the core. The variation in the amount of a first coating between tablets is preferably not more than 10% (between 90% and 110%), based on the total weight of the first coating. More preferred, the variation in the amount of a first coating is not more than 5% (between 95% and 105%), based on the total weight of the first coating. Factors (process conditions) that may influence the intra- and inter-tablet uniformity of the first coating include, for example, pan speed, spray rate, spray pattern, nozzle type, viscosity, drying temperature, air flow rate and coating time, as is known to the skilled person. When required, a temperature controlled curing step, for example heat treatment at 60-80° C. for 1-3 hours, is applied to the first coating after application, preferably spraying, of the first coating. In addition, the amounts of the water-soluble and/or water-insoluble hydrophilic substance in the first coating, and the identity of the water-soluble and/or water-insoluble hydrophilic substance, further provide means to modulate the timing of release of a first active ingredient. For example, a tablet comprising a pressed core and a first coating with an average thickness of about 35 micrometer, the coating comprising Ethocel 20 and lactose in a 3:2 ratio, provides release of the first active ingredient at about 36 minutes after hydration of the tablet, while the same composition of a tablet with a first coating with an average thickness of about 50 micrometer, provides release of the first active ingredient at about 84 minutes after hydration of the tablet. A tablet comprising a pressed core and a first coating with an average thickness of about 90 micrometer, the coating comprising Ethocel 20 and Avicell PH102 in a 3:2 ratio, provides release of the first active ingredient at about 105 minutes after hydration of the tablet. The skilled person is able to generate a delayed immediate release drug delivery system according to the disclosure, based on the teaching and the examples provided in this application.

Preferably, the dual release drug delivery system according to the disclosure is a tablet. The term “tablet” encompasses a “capsule” and a “caplet.” The total weight of a drug delivery system according to the disclosure is preferably at least 50 milligram, more preferred at least 150 milligram, and preferably is between 50 and 500 milligram, more preferred between 150 and 400 milligram, more preferred between 300 and 400 milligram, such as about 301.5 milligram, 325 milligram, or about 340 milligram. A dual release drug delivery system according to the disclosure provides delayed immediate release of the first active ingredient after about a predetermined period of time (lag time), about 2 hours, more preferred after about 2.5 hours, for example about 3 hours, after administration of the drug delivery system. The delayed release of the first active ingredient is also within about 6 hours, preferably within about 5 hours, more preferred within about 4 hours, for example within about 3.5 hours, after administration of the drug delivery system. In some embodiments of the disclosure, the delayed release of the first active ingredient is an immediate release occurred between 2.5 to 5 hours, more preferred between 2.5 to 4 hours, more preferred between 2.5 to 3.5 hours, after administration of the drug delivery system.

The term “time controlled” drug delivery system refers to a drug delivery system that provides release of a first active ingredient after a predetermined period of time, for example 2 hours, whereby the release is independent of pH. The predetermined period of time is set and not dependent on the pH history in the gastro-intestinal tract.

The term “immediate release” drug delivery system refers to a drug delivery system that provides release of a substantial amount of a first active ingredient within a predefined period of time. An immediate release drug delivery system, for example, provides the release of more than 60% of a first active ingredient, more preferred more than 70%, more preferred more than 80%, within 30 minutes after rupture of the coating, more preferred within 20 minutes, more preferred within 8 minutes after rupture of the coating.

Methods and means to determine the amount of a first active ingredient that is released from a drug delivery system, and the time frame within which the ingredient is released, such as for example compendial dissolution methods, are known to the skilled person such as, for example, United States Pharmacopoeia (USP) dissolution tests based on Apparatus 2 (the paddle method) and Apparatus 3 (the reciprocating cylinder).

The immediate release of a first active ingredient is thought to be caused by moisture induced stress relaxation. The driving force for this stress relaxation is the amount of stored energy within the core as surrounded by the polymer coating (Van der Voort Maarschalk et al., 1997. Int J Pharmaceutics 151: 27-34; Van der Voort Maarschalk et al., 1997. Pharm Res 14: 415-419; Steendam et al., 2001. J Control Rel 70: 71-82; Laity and Cameron, 2010. Eur J Pharm Biopharm 75: 263-276). Stress relaxation mediates the breakage of a coated core according to the disclosure in a nonlinear fashion. Hydration of the core and the hydrophilic substance in the first coating mediates stress relaxation such that an immediate burst of the coating after a predetermined period of time is obtained. It was found that the presence of more than 6% (w/w) of a water-insoluble, gel-forming disintegrant interferes with the immediate release of a first active ingredient and leads to more sustained release properties.

The dual release drug delivery system comprises a second coating surrounding the first coating. The second coating comprises a second active ingredient.

A second coating provides release of the second active ingredient in an immediate release fashion. The second coating may be pressed or sprayed onto the outer surface of the first coating. Methods for pressing or spraying are known in the art. A second coating that surrounds the first coating advantageously protects the integrity of the first coating, for example during packaging or storage of a dual drug delivery system. This will preferably decrease or minimize damage to the first coating occurring during packaging or storage that might affect the lag time of the release of the first active ingredient from the core of the dual drug delivery system.

The second coating is preferably sprayed onto the outer surface of the first coating. When a spray coat is used it is generally formulated to contain a drug and film forming ingredient so that the drug is dispersed in the film that overlays the first coating of the core. Such film forming ingredients are known in the art and may be for example hydroxypropylmethylcellulose, povidone, hydroxyethylcellulose, other modified celluloses known in the art, polyacrylates, polymethacrylates, and polymethyl/ethylmethacrylates. A film forming ingredient according to the disclosure preferably comprises hydroxypropylmethylcellulose, more preferred low molecular weight hydroxypropylmethylcellulose with a number average molecular weight below 20,000; more preferred below 10,000. The spray coat is formulated to give an immediate release by forming a coat that dissolves quickly. The amount of a film-forming ingredient is preferably between 0.05 and 40% (w/w), based on the total weight of the second coating, more preferred between 1 and 30% (w/w) such as, for example, about 20% (w/w).

The second coating preferably comprises a weight of between 0.5 and 5% (w/w) based on the total weight of the drug delivery system. Preferably the coating comprises a weight of between 1% and 3% and preferably between 1,5 and 2,5% (w/w) based on the total weight of the drug delivery system. In a preferred embodiment the second coating of a drug delivery system comprises a weight of between about 1-20 mg per unit. Preferably the second coating comprises a weight of about 3-15 mg per unit. In a particularly preferred embodiment the second coating of a drug delivery system of the disclosure comprises a weight of about 4-10 mg per unit.

The second coating of a dual drug delivery system according to the disclosure comprises a second active ingredient. The amount of a second coating that is sprayed onto the outer surface of the first coating therefore determines the amount of the second active ingredient in the dual drug delivery system. The amount of a second coating, therefore, needs to be controlled. The variation in the amount of a second coating between tablets is preferably not more than 10% (between 90% and 110%), based on the total weight of the second coating. More preferred, the variation in the amount of a second coating is not more than 5% (between 95% and 105%), based on the total weight of the second coating. Factors (process conditions) that may influence the intra- and inter-tablet uniformity of the second coating include, for example, pan speed, spray rate, spray pattern, nozzle type, viscosity, drying temperature, air flow rate and coating time, as is known to the skilled person. The amount of a second active ingredient is preferably between 0.05 and 20% (w/w), based on the total weight of the second coating, more preferred between 0.5 and 10% (w/w).

Examples of known excipients that may be added to a sprayed or pressed second coating for controlled release are one or more polymers or copolymers selected from acrylic and methacrylic acid polymers and copolymers such as acrylic acid and methacrylic acid copolymers, methyl methacrylate copolymers, ethoxyethyl methacrylates, cyanoethyl methacrylate, poly(acrylic acid), poly(methacrylic acid), methacrylic acid alkylamide copolymer, poly(methyl methacrylate), polymethacrylate, poly(methyl methacrylate) copolymer, polyacrylamide, aminoalkyl methacrylate copolymer, poly(methacrylic acid anhydride), glycidyl methacrylate copolymers and ethylcellulose. The amount of known excipients is preferably below 10% (w/w), based on the total weight of the second coating, more preferred below 5% (w/w), more preferred below 1% (w/w).

The second coating of a dual drug delivery system according to the disclosure provides immediate delivery of the second active ingredient in the mouth. The term “mouth” comprises the interspace between the lips and the teeth, the interspace between the cheek and the teeth, the oral cavity which is delimited by the palate and tongue and the sublingual area. The second active ingredient is preferably released in the sublingual space in the mouth. The term “immediate release of the second active ingredient” refers to the rapid dissolution of the second coating in the mouth such that the second active ingredient is completely or substantially completely released within a short time frame within the mouth. The term “immediate release of the second ingredient” indicates that at least 50% of the second active ingredient is released within 5 minutes, more preferred within 4 minutes, more preferred within 3 minutes, more preferred within 2 minutes, most preferred within 1 minute, for example within 90 seconds, after oral administration of the dual drug delivery system. It is more preferred that at least 70% of the second active ingredient is released within 5 minutes, more preferred within 4 minutes, more preferred within 3 minutes, more preferred within 2 minutes, most preferred within 1 minute after oral administration of the dual drug delivery system. An advantage of a dual drug delivery system according to the disclosure is that food-effects are minimized. The term “food-effects” refers to the difference in the rate and extent of absorption of a drug that is administered shortly after a meal (fed conditions), as compared to administration under fasting conditions. The release of the first active ingredient is not dependent on the pH and therefore not likely to be influenced by food effects. In addition, the formulation of the second active ingredient as an immediate release formulation also minimizes food-effects for the release of the second active ingredient. A further advantage of a dual drug delivery system according to the disclosure is that it provides two independent dosing routes in one tablet.

A further advantage of a dual drug delivery system according to the disclosure is that it provides first-pass free absorption into the systemic circulation of one active ingredient (defined herein as second active ingredient) in combination with gastro-intestinal absorption of a further active ingredient (defined herein as first active ingredient) in one drug delivery system.

The second active ingredient is testosterone or a functional analogue or derivative thereof. Testosterone is also known under the chemical name 17-[beta]-hydroxyandrost-4-en-3-one which can be obtained in various ways: it may be isolated and purified from nature or synthetically produced by any manner. This active ingredient is preferably used in a therapy for treatment of male or female sexual dysfunction, desire dysfunction, or erectile dysfunction, and preferably for the treatment of Hypoactive Sexual Desire Disorder.

The term “testosterone or functional analogue or derivative thereof” refers to testosterone or a precursor or metabolite of testosterone that provides the same or a similar function as testosterone. Preferred precursors of testosterone are selected from pregnenolone, 17α-hydroxypregnenolone, progesterone,17α-hydroxyprogesterone, dehydroepiandrosterone, androstenedione, and androstenediol. Preferred metabolites of testosterone are selected from hydroxyandrostenedione, hydroxytestosterone, including 2β-, 6β-, 7α-, 12α-, and 16α-hydroxytestosterone, and dihydrotestosterone, including 5 a- and 5β-dihydrotestosterone. A preferred analogue of testosterone is capable of binding to an androgen receptor. It is most preferred that the testosterone or a functional analogue or derivative thereof is testosterone.

According to the disclosure, although it is not considered bound by theory, an effect on the central nervous system and the peripheral system are required, whereby the signal to the central system is provided by testosterone or a functional analogue or derivative thereof (having the same kind of activity) and the peripheral signal is provided by the PDE5-inhibitor.

Testosterone in the circulation is typically bound by SHBG (steroid hormone binding globulin) and by albumin. Without wishing to be bound by theory, the dose of testosterone according to the drug delivery system of the disclosure is high enough to saturate the albumin and SHBG (i.e., the concentration of testosterone is high enough to overcome complete binding of testosterone by SHBG or albumin) such that at least a fraction of testosterone not bound by albumin and SHBG is present in the blood (free testosterone).

Optionally, the level of free testosterone should be a peak serum level of free testosterone of from 0.01 to 0.1 ng/mL. Preferably, the peak serum free testosterone level is from 0.025 to 0.6 ng/mL, for example from 0.025 to 0.5 ng/mL. Alternatively, the level of peak free testosterone may be between 0.3-2%, preferably from 0.5-2%, of peak total serum testosterone. The peak of serum free testosterone level will typically occur between 5 and 30 minutes after administration of the testosterone. Preferably, the plasma/serum total concentration of the testosterone peaks within 1 hour, preferably within 30 minutes, after administration (of the testosterone).

Testosterone is preferably given in a formulation wherein there is a (short-lasting) (high, i.e., 10-100 times increase of normal testosterone serum levels) peak of testosterone in the blood circulation of the subject to whom it is administered. The term “short-lasting” means that there is a sharp increase of the serum testosterone level and approximately 5-30 minutes after administration a peak serum level of testosterone is obtained. The peak serum level sharply decreases and after approximately 120 minutes the testosterone serum level is back to the level before testosterone administration.

About 3 to 6 hours after this plasma testosterone peak, there is a testosterone effect peak, i.e., there is a time lag in the effect of testosterone on genital arousal in sexually functional women. The delayed release of the first ingredient of the disclosure is an immediate release occurred between 2 to 6 hours, preferably between 2.5 to 3.5 hours, after the release of the second active ingredient (testosterone, or a functional analogue or derivative thereof). The plasma concentration of the first active ingredient peaks between 3 to 6 hours, preferably between 3 to 5 hours, for example between 3 to 4 hours, after the administration of the drug delivery system of the disclosure. It is clear to the skilled person that the compound capable of at least in part inhibiting the interaction between a GAF-A domain and cGMP (for example a GAF-A blocker) must be dosed and released in such a way that its effect at least partly coincides with the effect of testosterone, i.e., the effect of the compounds must be present 3.5-5.5 hours after the induced plasma peak in testosterone (or an analogue thereof). The skilled person can, based on the pharmacological kinetics of the compound, easily determine a suitable formulation as well as a suitable concentration for the compounds.

Preferably, the dual release drug delivery system comprises the second active ingredient in amorphous form in an amount of from 0.4 to 1.5 mg, more preferably 0.4 to 1.0 mg. In a preferred embodiment of the disclosure, the dual release drug delivery system comprises equivalent to 0.5 mg the second active ingredient. In an alternative embodiment of the disclosure, the dual release drug delivery system comprises equivalent to 1.0 mg the second active ingredient. Alternatively yet still preferably, the dual release drug delivery system comprises the second active ingredient in amorphous form in an amount no less than 1.0 mg, for example 1-5 mg, preferably 1-4 mg, for example in an amount of 2.0 mg, 2.5 mg, 3.0 mg, 3.5 mg, 4.0 mg. The inventors of the disclosure have found that, surprisingly, a drug delivery system comprising testosterone at an amount of at least equivalent to 0.4 mg gives an optimal free testosterone peak level that enhances female sexual desire, when administered in combination with a PDE-5 inhibitor released between 2-6 hours after the administration.

Optionally, the second coating may comprise a mixture of the second active ingredient in amorphous form in an amount of from about 0.5 to 1.0 mg or no less than 1.0 mg, for example 1-4 mg; a coating polymer in an amount of between about 0.25-25 mg; and water in an amount of between about 0.0-10% w/w of the second coating. Alternatively, the second coating may comprise a cyclodextrin or a polyvinylpyrolidone or a combination thereof, in an amount of between 0.25-25 mg. In some embodiments, the second coating comprises a mixture of the second active ingredient in amorphous form in an amount of between about 0.5 to 1.0 mg; a cyclodextrin or a polyvinylpyrolidone or a combination thereof in an amount of between about 0.5-12.5 mg; and water in an amount of between about 0.0-5% w/w of the second coating. The combination of cyclodextrin and polyvinylpyrolidone helps to improve the stability of the second active ingredient (testosterone or a functional analogue or derivative thereof) in drug delivery systems, in particular in tablets. Both cyclodextrin and polyvinylpyrolidone prevent amorphous testosterone or a functional analogue thereof from crystallizing in the coating when exposed to prolonged incubation and/or various temperatures such as can occur during storage of the drug delivery system.

Preferably, the second coating further comprises a carrier selected from hydroxypropyl-beta-cyclodextrin, poly-beta-cyclodextrin, gamma-cyclodextrin and polyvinylpyrolidone. A preferred polyvinylpyrolidone is low molecular weight polyvinylpyrolidone with a molecular weight of maximal 80000. A suitable polyvinylpyrolidone is preferably selected from K10, K15, K25, K30, and K50. A most preferred carrier is hydroxypropyl-beta-cyclodextrine. The presence of a poorly soluble steroid such as testosterone and a carrier such as a cyclodextrin provides rapid and efficient delivery of the testosterone to the mucous membrane, from which the testosterone is then rapidly absorbed into the circulation. The amount of the carrier is preferably between 0.5 and 70% (w/w), based on the total weight of the second coating, more preferred between 2 and 60% (w/w), more preferred between 5 and 50% (w/w).

In one embodiment of the disclosure, the second coating comprises 0.5 mg testosterone and 5 mg hydroxypropyl-beta-cyclodextrines (carrier). In another embodiment of the disclosure, the second coating comprises 1.0 mg, 1.5 mg, 2.0 mg, 2.5 mg, 3.0 mg, 3.5 mg or 4.0 mg testosterone and 5 mg hydroxypropyl-beta-cyclodextrines (carrier).

The second coating preferably comprises a flavoring compound in addition to the second active ingredient and one or more excipients, such as, for example, a coloring agent. The flavoring compound may be any natural, artificial or synthetic compound or mixture of compounds that is pharmaceutically acceptable. An illustrative list of flavors for pharmaceutical applications includes cyclic alcohols, volatile oils, synthetic flavor oils, flavoring aromatics, oils, liquids, oleoresins and extracts derived from plants, leaves, flowers, fruits, stems, roots, and combinations thereof. Non-limiting examples of cyclic alcohols include menthol, isomenthol, neomenthol and neoisomenthol. Non-limiting examples of flavor oils include spearmint oil, cinnamon oil, oil of wintergreen (methyl salicylate), peppermint oil, clove oil, bay oil, anise oil, eucalyptus oil, thyme oil, cedar leaf oil, oil of nutmeg, allspice, oil of sage, mace, oil of bitter almonds, cassia oil, and combinations thereof. Suitable flavors also include, for example, artificial, natural and synthetic fruit flavors such as citrus oils (e.g., lemon, orange, lime, and grapefruit), fruit essences (e.g., lemon, orange, lime, grapefruit, apple, pear, peach, grape, strawberry, raspberry, cherry, plum, pineapple, apricot or other fruit flavors). Other useful artificial, natural and synthetic flavors include sugars, polyols such as sugar alcohols, artificial sweeteners such as aspartame, stevia, sucralose, neotame, acesulfame potassium, and saccharin, chocolate, coffee, vanilla, honey powders, and combinations thereof. Other useful flavors include aldehydes and esters, such as benzaldehyde (cherry, almond), citral (lemon, lime), neral (lemon, lime), decanal (orange, lemon), aldehyde C-8 (citrus fruits), aldehyde C-9 (citrus fruits), aldehyde C-12 (citrus fruits), tolylaldehyde (cherry, almond), 2,6-dimethyloctanal (green fruit), 2-dodenal (citrus mandarin), and combinations thereof. A preferred flavoring compound is a cyclic alcohol such as, for example, menthol, isomenthol, neomenthol and neoisomenthol, preferably combined with an artificial sweetener such as aspartame. The amount of a flavoring compound is preferably between 0.1 and 60% (w/w), based on the total weight of the second coating, more preferred between 1 and 40% (w/w).

The presence of a flavoring compound in the second coating of a dual drug delivery system according to the disclosure may mask a bitter or objectional-tasting drug or excipient.

It is preferred that the flavoring compound in the second coating of a dual release drug delivery system according to the disclosure rapidly disappears from the oral cavity. Sensing of the particular flavor in the oral cavity indicates to the user that the second coating has not completely dissolved and that drug delivery system which is encompassed within the second coating is to be held in the mouth. During use, when the second active ingredient is co-delivered with a flavoring compound from the second coating, the subject can easily recognize that the system is delivering the second active ingredient due to the presence of the flavor (taste). Eventually, the entire dose of second active ingredient is delivered. At this point, the system may also stop delivering the flavor. The disappearance of the flavor (taste) indicates that the drug delivery system may be swallowed.

The skilled person will understand that a flavoring compound may be present in the first coating, instead of in the second coating. In that case, the appearance of the flavor (taste) indicates that the drug delivery system may be swallowed. The skilled person will further understand that a first flavoring compound may be present in the second coating, while a second flavoring compound is present in the first coating. Upon disappearance of the first flavor (taste), and tasting of the second flavor (taste), the subject knows that the system has delivered the entire dose of the second active ingredient.

It is further preferred that the roughness of the outer surface of the second coating differs from the roughness of the outer surface of the first coating in a system according to the disclosure. A subject can be instructed to swallow the time controlled, immediate release drug delivery system when a difference in roughness becomes evident. This provides sufficient retention time of a system according to the disclosure in the mouth so that the second active ingredient is sufficiently released and absorbed.

The disclosure further provides the use of a flavoring compound in a dual delivery drug system, for indicating that the system is to be held in the mouth until the flavor (taste) has disappeared.

The disclosure further provides the use of a flavoring compound in a dual delivery drug system, for indicating that the system is to be held in the mouth until the flavor (taste) appears.

The disclosure further provides a method for preparing a dual release delivery drug system comprising a first and a second coating, whereby a flavoring compound is present in the second coating for indicating that the system is to be held in the mouth until the flavor (taste) has disappeared.

The disclosure further provides a method for preparing a dual release delivery drug system comprising a first and a second coating, whereby a flavoring compound is present in the first coating for indicating that the system is to be held in the mouth until the flavor (taste) appears.

The disclosure further provides the use of a difference in roughness between an outer surface of a first coating and an outer surface of a second coating in a dual release drug delivery system for indicating that the system is to be held in the mouth.

The disclosure further provides the use of a difference in roughness between an outer surface of a first coating and an outer surface of a second coating in a dual release drug delivery system for indicating that the system is to be swallowed.

The disclosure further provides a method for preparing a dual release drug delivery system comprising a first and a second coating, wherein a roughness of an outer surface of the first coating differs from a roughness of an outer surface of the second coating.

Preferably, the first active ingredient is to be administered orally. Preferably, the second active ingredient is to be administered sublingually. A further advantage of a dual drug delivery system according to the disclosure is that it provides sublingual absorption into the systemic circulation of one active ingredient (defined herein as second active ingredient) in combination with gastro-intestinal absorption of a further active ingredient (defined herein as first active ingredient) in one drug delivery system.

In the disclosure it was found that the second active ingredient is very well absorbed by the mucosa in the mouth. The absolute absorption as measured by bioavailability and the rate of absorption were significantly better when compared to a liquid with the same amount of active ingredient. Both variables were measured by measuring the concentration of the active ingredient in the blood of the recipient at different time points after administration.

The disclosure therefore provides a use, wherein the testosterone or an analogue thereof is provided, preferably in the form of a sublingual formulation, optionally comprising cyclodextrins as carrier.

For the disclosure the routes of administration of choice are those which are the least invasive (for example oral, buco-mucosal or intranasal), so as to cause the least negative impact on female sexual desire.

A typical example of a formulation is given in hydroxypropyl-beta-cyclodextrin, but other beta cyclodextrins and other usual excipients, diluents and the like are within the skill of the art for preparing a formulation comprising testosterone or a functional analogue or derivative thereof, which releases essentially all of the testosterone within one short burst. The burst will typically be within a short time interval (for example within 60-120 seconds, more preferably within 60 seconds) upon administration, leading to blood peak levels of testosterone about 5-30 minutes later.

The disclosure further provides a method for enhancing female sexual desire in the treatment of Female Sexual Interest and Arousal Disorder (FSIAD). The method comprises administering the dual release drug delivery system for use according to the disclosure to a subject. Optionally, the drug delivery system may be taken approximately 3-4 hours before planned or anticipated sexual activity.

Preferably, the administration comprises holding the dual release drug delivery system under the tongue for a period of time between 10 seconds and 5 minutes, more preferred for at least 30 seconds, for example between 30 seconds and 2.5 minutes, more preferred for at least 60 seconds, for example between 60 seconds and 90 seconds, or for at least 90 seconds, prior to swallowing it.

In a particularly preferred embodiment, the drug release system is placed under the tongue, whereupon the individual gently holds or moves such as swishes, the drug release system about for 90 seconds. It is preferred that the individual does not swallow the drug release system or saliva during the incubation period in the mouth and preferably under the tongue. The individual preferably does not chew or bite on the drug release system. Upon completion of the incubation time, the drug release system is preferably swallowed as a whole by the individual, optionally together with a fluid such as water.

To create a more permanent psychological change, the method may be preferably augmented by an “approach induction” treatment, during which the central and bodily processes activated by the first and second active ingredients under sexually relevant stimulation are perceived and become associated with a positive hedonic tone or with activation of the behavioral approach system. The perception of bodily reactions by focusing attention on genital arousal is made possible by testosterone (whereby the genital arousal is synergistically enhanced by the PDE-5 inhibitor) and can be emphasized by verbal instructions. These positively motivated stimuli consist of pictures of happy faces of persons of the patient's sexually preferred gender, possibly including the face of the partner. The pictures of the faces are presented subliminally, so that in an unobtrusive way the behavioral approach system becomes activated.

The disclosure also provides a kit of parts comprising a dual release drug delivery system as described herein. Optionally, the kit further comprises instructions in respect to the administration of the drug delivery system. The kit may, optionally, further comprise a compound capable of competing with testosterone (or a functional analogue or derivative thereof) for SHBG binding.

The kit is useful for any individual suffering from reduced female sexual desire, be it through psychological or physiological causes or combinations thereof. It is thus also useful for subjects having Female Sexual Interest and Arousal Disorder (FSIAD), in particular acquired FSIAD.

A preferred dual release drug delivery system according to the disclosure comprises:

### Core:

between 100 mg and 150 mg, preferably between 109 mg and 126.5 mg, of Pharmacel pH102;
between 100 mg and 150 mg, preferably between 109 mg and 126.5 mg, of DicalciumPhosphate 0 aq;
between 20 mg and 100 mg, preferably between 20 mg and 70 mg, of Sildenafil citrate;
between 10 mg and 20 mg, preferably about 12 mg of Croscarmellose;
between 1 mg and 2 mg, preferably about 1.5 mg of Magnesiumstearate;

### First Coating

between 5 mg and 20 mg, preferably about 12.5 mg of Ethocel 20;
between 5 mg and 20 mg, preferably about 12.5 mg of Avicel pH 105;

### Second Coating:

between 1 mg and 2 mg, preferably about 1.34 mg of HPMC 5cps;
between 2 mg and 3.5 mg, preferably about 2.66 mg of HydroxyPropyl-beta-cyclodextrin;
between 0.3 mg and 1.5 mg, preferably between 0.4 and 1.0 mg of Testosterone.

The second coating of the preferred dual drug delivery preferably further comprises between 1 mg and 2 mg, preferably about 1.34 mg of Peppermint-oil and between 0.5 mg and 1.5 mg, preferably about 1.0 mg of Aspartame.

According to a further aspect, the disclosure also provides a method for identifying a patient suitable for receiving a combination treatment with higher testosterone dosage (no less than 1.0 mg) in the treatment of FSIAD. According to the method, the patient is identified as a suitable candidate when the patient has one of the following impact factors:
1) The patient is postmenopausal;
2) The patient's BMI is no greater than a pre-determined threshold, for example 25, 24.5, 24, 23.5, 23, or preferably 22.5;
3) The patient is using COCs or receiving HRT;
4) The patient has a plasma SHBG level no lower than a pre-determined threshold, for example 80 nmol/L, 90 nmol/L, or preferably 100 nmol/L; or
5) the patient also has a plasma albumin level no greater than a pre-determined threshold, for example 45 g/L, 40 g/L, or preferably 37 g/L.

As unexpectedly found by the inventors of the disclosure, and as shown in the Examples described herein, the impact factors recited above can provide good indication on the dosage of testosterone required in order to achieve good efficacy in the treatment of FSIAD whilst avoiding any (serious) side effects. The disclosure offers a simple yet effective way that helps to identify patients with different medication requirements at an early stage, thereby enhancing the chance of success with treatments and improving patient's confidence levels. The impact factors can also interact with each other to further drive up requirements on testosterone. Patients with multiple impact factors may require further increase on the testosterone dosage in order to achieve satisfactory efficacy. For example, patients with one impact factor may require a combination therapy with at least 1-2 mg testosterone. Testosterone dosage in the combination therapy can go even higher, for example to at least 4 mg, for patients whose BMI is extremely low (for example lower than 20, lower than 18, or lower than 16; or patients whose plasma SHBG levels are very high (for example more than 20% higher, more than 30% higher, more than 40% higher, or more than 50% higher, than the pre-determined threshold.

The disclosure also provides a method for determining the testosterone dosage in a combination therapy (with a PDE5 inhibitor such as sildenafil) for a patient in the treatment of FSIAD. According to the method, the testosterone dosage in the combination therapy is determined based on the number of impact factors the patient has. Patients with at least one impact factor will start with a combination therapy comprising at least 1.0-2.0 mg testosterone. Using this method, a medical practitioner can quickly choose a combination therapy that is likely to work for a particular patient, based on the patient's demographic data or blood test results, broadening the window of opportunity for achieving desirable efficacy.

Preferably, the testosterone is administered in a way that the testosterone is immediately bio-available upon administration. In other words, the testosterone is administered in a “immediate release” format.

The term “immediate release” refers to a release of a substantial amount of an active ingredient within a predefined period of time. An immediate release, for example, provides the release of more than 60% of the ingredient, more preferred more than 70%, more preferred more than 80%, within 30 minutes after rupture of the coating, more preferred within 20 minutes, more preferred within 8 minutes upon administration or after rupture of the separating coating, when present.

Preferably, the testosterone is administered orally, for example, released in the sublingual space in the mouth. For example, the testosterone may be administered as a sublingual tablet or oral solution/liquid. It may be applied some time (for example 120 seconds, 90 seconds, or 60 seconds) before swallowing. Alternatively, the testosterone may be administered as a sublingual or oral inhalation or intranasal inhalation spray.

Due to the abundant vascularization in mouth, including the sublingual space in the mouth, rapid adsorption of testosterone is possible, helping to bring a rapid (high) peak of (bioavailable) testosterone in the bloodstream. The abundance of bioavailable testosterone sends a signal to the central system, and getting the body ready for the stimulation from the PDE5 inhibitor.

The patient receiving a high testosterone combination therapy may be postmenopausal. Menopause significantly affects various hormone and protein levels in the body. For example postmenopausal women may see drops in estrogen, progesterone, and testosterone levels. The level of SHBG in menopausal women may also decrease. Nevertheless, high SHBG levels have also been reported in postmenopausal women, probably related to hormonal changes due to weight increase and/or use of HRT. It has been found in clinical trials that, surprisingly, patients in the treatment of FSIAD who are postmenopausal appeared to have benefited from high testosterone levels.

As hormone and/or SHBG levels can also be affected by medications such as combined oral contraceptives (COCs) or combined hormone replacement therapy (HRT), patients who are using COCs or receiving HRT are also considered suitable for a high testosterone dosage in the combination therapy with a PDE5 inhibitor in the treatment of FSIAD.

In addition, key general health indicators such as BMI also affect hormone levels. For example, insufficient body fat can lead to decreased hormone levels (such as estrogen, testosterone, thyroid hormones etc.) as adipose (body fat) tissues contribute to the production of hormones. Low BMI, particularly when associated with low body fat and muscle mass, can also lead to low insulin levels and potentially impact blood sugar regulation, which in turn effects hormone levels and balance. The inventors of the disclosure found that, surprisingly, patients with low BMI, for example a BMI no greater than 25, 24.5, 24, 23.5, 23, preferably a BMI no greater than 22.5, or even lower than 20, require testosterone dosage of no less than 1.0 mg, for example 1.0-4.0 mg, in combination therapy for the treatment of FSIAD, for getting desired efficacy.

SHBG and albumin are important proteins that bind to testosterone, thereby affecting the level of bio-available testosterone in the bloodstream, that are ready for biological activities. High SHBG level or low albumin level is indicative of in vivo allocation and activities of testosterone upon administration. Increasing the testosterone dosage to no less than 1.0 mg, for example 1.0 to 4.0 mg, in a combination therapy for the treatment of FSIAD for patients with high plasma SHBG level and/or low plasma albumin level has shown increased efficacy. A plasma SHBG level is considered “high” if it's at least 80 nmol/L, 90 nmol/L, preferably at least 100 nmol/L, and, for example if it's 80-250 nmol/L, or 100-200 nmol/L. A plasma albumin level is considered “low” if it's no more than 45 g/L, 40 g/L, or preferably 37 g/L, even more preferably, and, for example if it's 10-40 g/L, or 15-35 g/L.

The combination therapy may comprise the PDE5 inhibitor in an amount sufficient for achieving the desired efficacy with low side effects. Preferably the PDE5 inhibitor is sildenafil (or a salt thereof), and the amount of sildenafil may be from 25 to 120 mg, for example 25 mg, 50 mg, 75 mg or 100 mg.

The combination therapy comprising (high dosage) testosterone and PDE5 inhibitor may be administered as one formulation or as separate formulations. The formulation or the separate formulations are such that testosterone is released as an immediate release and the PDE5 inhibitor is released as a delayed immediate release, and the delay is preferably 2-6 hours, most preferably 2.5-3.5 hours (after the release of testosterone).

The method of the disclosure may be further personalized. For example, the method may further comprises further personalization or adjustment of the dosage of testosterone and/or the PDE5 inhibitor such as sildenafil, based on initial evaluation of efficacy and side effects after treating the patient with the chosen dosage/pharmaceutical composition for a pre-determined period of time. For example, if, according to the evaluation, the efficacy of the treatment is not sufficient, the dosage of testosterone may be further increased, for example increased by 0.5 mg or 1.0 mg. The additional testosterone can be administered in the form of an additional formulation, for example a separate formulation comprising 0.5 mg or 1.0 mg testosterone. Or the patient can take a combined formulation comprising the higher testosterone dosage (in combination with the desired dosage of PDE5 inhibitor). For example, the patient may administer a combination therapy comprising 1 mg testosterone and a PDE5 inhibitor, and a further pharmaceutical composition comprising 1.0 mg testosterone, for example an oral 1.0 mg testosterone composition, preferably as a liquid or sublingual administration, or a 1.0 mg testosterone composition in a solid dosage form such as a tablet, a capsule, or in a dosage form suitable for oral or intranasal inhalation. Alternatively, if the side effects of the treatment are too severe, the dosage of the PDE5 may be reduced. When the dosage of the PDE5 inhibitor is adjusted, the dosage of testosterone can be adjusted at the same time to 0.5 mg or to 1.5 mg. For example, the adjustment may comprise giving the patient a pharmaceutical composition comprising 0.5 mg testosterone and 50 mg sildenafil, optionally in combination with a further pharmaceutical composition comprising 1.0 mg testosterone, preferably as a liquid or sublingual administration, for example an oral 1.0 mg testosterone composition, or a 1.0 mg testosterone composition in a solid dosage form such as a tablet, a capsule, or in a dosage form suitable for oral or intranasal inhalation.

The method and composition of the disclosure offers great flexibility in terms of tailoring it for individual needs.

The pre-determined treatment period may be any period sufficient to establish both the efficacy and side effects of the treatment. For example, the pre-determined treatment period may be 4 weeks or 8 weeks, preferably 8 weeks.

According to a further aspect, the disclosure provides a combination therapy (a pharmaceutical composition) comprising a steroid such as testosterone or a functional analogue or derivative thereof, in combination with a PDE5 inhibitor, preferably for use for the patient identified by the method of the disclosure. The testosterone or a functional analogue or derivative thereof is present in an amount equivalent to no less than 1 mg, for example 1-5 mg, preferably 1-4 mg, testosterone. Based on earlier clinical studies it has been observed that 0.5 mg of testosterone may not be as efficacious in women with at least one of the following characteristics: post-menopausal, use of COC or receive HRT, high SHBG, low albumin and (very) low BMI. Women with one of these characteristics may typically benefit from a higher dose of testosterone, e.g., 1-2 mg. Women who have at least two of these characteristics may benefit from an even higher dose, such as 3-4 mg.

The combination therapy of the disclosure is useful for enhancing female sexual desire in the treatment of FSIAD, in particular acquired FSIAD. The inventors of the disclosure have found that when used for enhancing female sexual desire in the treatment of FSIAD, testosterone and the PDE5 inhibitor can synergistically enhance both central and peripheral processes. In particular, the high dose (no less than 1 mg) testosterone in the pharmaceutical composition of the disclosure provides sufficient bioavailable testosterone in the bloodstream even in patients with relatively high SHBG level (thus high proportion of strongly bound testosterone) or relatively low albumin level (thus low proportion of weakly bound testosterone) due to, for example, their general health conditions, use of COC or HRT, or menopausal status).

The combination therapy of the disclosure may also be useful in alternative therapies such as psychotherapy and counselling, in particular with regards to relationship or couple therapy. Approximately 30-50% of couples in therapy report female sexual issues, and addressing female sexual issues in therapy often involves a multidisciplinary approach, including improving communication, exploring emotional and psychological factors, and incorporating medical interventions. The combination therapy of the disclosure provides a tool for improving sexual relationship between couples and partners, and can increase chance of success and/or improve the outcome of psychotherapy and counselling.

The pre-determined thresholds (referred to hereabove and hereafter) represents reference points for each of those parameters. The skilled person will be able to determine a suitable threshold, based on common general knowledge and the teaching of the current application, using means and methods already known in the art.

In the context of the current application, the term “testosterone” is used to also encompass a functional analogue or derivative thereof, where it is not explicitly mentioned. The amount of testosterone specified in the current application is to be understood to refer to the “equivalent testosterone” amount, when a functional analogue or derivative of testosterone is used. Testosterone is also known under the chemical name 17-[beta]-hydroxyandrost-4-en-3-one which can be obtained in various ways: it may be isolated and purified from nature or synthetically produced by any manner.

The term “testosterone or functional analogue or derivative thereof” refers to testosterone or a precursor or metabolite of testosterone that provides the same or a similar function as testosterone. Preferred precursors of testosterone are selected from pregnenolone, 17α-hydroxypregnenolone, progesterone,17α-hydroxyprogesterone, dehydroepiandrosterone, androstenedione, and androstenediol. Preferred metabolites of testosterone are selected from hydroxyandrostenedione, hydroxytestosterone, including 2β-, 6β-, 7α-, 12α-, and 16α-hydroxytestosterone, and dihydrotestosterone, including 5 a- and 5β dihydrotestosterone. A preferred analogue of testosterone is capable of binding to an androgen receptor. It is most preferred that the testosterone or a functional analogue or derivative thereof is testosterone.

According to the disclosure, although it is not considered bound by theory, an effect on the central nervous system and the peripheral system are required, whereby the signal to the central system is provided by testosterone or a functional analogue or derivative thereof (having the same kind of activity) and the peripheral signal is provided by the PDE5-inhibitor.

Testosterone in the circulation is typically bound by SHBG and by albumin.

Albumin is the most abundant protein in human blood plasma. It plays a crucial role in maintaining oncotic pressure, which helps keep fluid within the blood vessels. Albumin also serves as a carrier protein, binding to various substances such as hormones, vitamins, and drugs, facilitating their transport through the bloodstream. In particular, albumin binds to testosterone with low affinity. Additionally, it has antioxidant properties and helps in maintaining pH balance. Low levels of albumin can indicate liver disease, kidney disease, or other medical conditions. Albumin is produced by the liver, and the production is influenced by including nutritional status, hormonal signals, and overall liver function.

The binding of testosterone to albumin is a dynamic process characterized by a rapid equilibrium. This binding is relatively weak and reversible, which means that albumin-bound testosterone can quickly dissociate and become available for cellular uptake, contributing to the bioavailable fraction of testosterone. In particular, the binding is non-covalent (e.g., by hydrophobic interactions and electrostatic forces) and quickly reaches a state of equilibrium, where the rates of binding and unbinding are balanced. The association (binding) and dissociation (unbinding) of testosterone with albumin occur rapidly. The exact time frame for these interactions is in the order of milliseconds to seconds. Due to the rapid and reversible nature of its binding, albumin-bound testosterone is considered part of the bioavailable pool of testosterone. This means it can quickly dissociate and become free to enter cells and exert biological effects. The rapid binding and release of testosterone from albumin help buffer fluctuations in hormone levels, ensuring a relatively stable concentration of free testosterone available to tissues.

Albumin is present in high concentrations in the blood. In healthy adults, the normal range for serum albumin is typically between 3.5 and 5.0 g/dL (grams per decilitre). Albumin levels can fluctuate slightly due to factors like hydration status, diet, and physical activity.

Factors that may affect albumin levels include:
Age. In younger adults, albumin levels are generally stable and within the normal range, assuming good health and adequate nutrition. As people age, serum albumin levels tend to gradually decline. This decline is not always indicative of disease but may reflect age-related changes in liver function, protein synthesis, and overall health status. Lower albumin levels in older adults are often associated with malnutrition, frailty, and chronic diseases. In many cases, hypoalbuminemia (low albumin levels) can be a marker for poor nutritional status or the presence of an underlying chronic illness. Some studies suggest that low albumin levels may be associated with an increased risk of cognitive decline and dementia in older adults, although the exact relationship remains under investigation. Albumin is also important for immune function, and low levels in older adults can be linked to weakened immune responses and increased susceptibility to infections.
Hormonal changes, for example menopause, or hormone replacement therapy. Some studies suggest that albumin levels may decrease slightly during menopause, partly due to hormonal changes and the associated effects on liver function. However, this decline is often subtle and may not be significant in healthy women without underlying conditions. Hormone replacement therapy (HRT), often used to manage menopausal symptoms, can influence albumin levels. Estrogen therapy, particularly oral estrogen, can increase the production of proteins in the liver, including SHBG and possibly albumin, though the effects on albumin are less pronounced. The overall impact of HRT on albumin levels may depend on the type and route of administration (e.g., oral vs. transdermal).
Certain medications commonly used by older adults, such as nonsteroidal anti-inflammatory drugs (NSAIDs) and corticosteroids, can affect albumin levels by influencing liver function or causing fluid retention.
Health conditions, for example BMI, liver function, hyperthyroidism, obesity, insulin resistance, and polycystic ovary syndrome (PCOS). A low BMI, particularly in the context of malnutrition or underweight status, is often associated with lower albumin levels. Malnutrition leads to inadequate protein intake, which can reduce albumin synthesis in the liver. This is commonly seen in individuals with chronic illnesses, eating disorders, or severe weight loss. Obesity (high BMI) can have complex effects on albumin levels. In some cases, individuals with higher BMI may have normal or even slightly elevated albumin levels, particularly if their nutritional status is adequate. However, obesity is often associated with conditions like insulin resistance, chronic inflammation, and fatty liver disease, which can alter albumin production. Many diseases, such as liver disease, kidney disease, heart disease, inflammatory and autoimmune diseases, cancer, etc., can also influence albumin level. Low albumin levels often signal poor health outcomes and can be used as a marker for disease severity, prognosis, and the need for medical intervention.

SHBG is a glycoprotein produced mainly by the liver. it binds to sex hormones, specifically testosterone and estradiol, which are the primary male and female sex hormones, respectively. By binding to testosterone and estradiol, SHBG controls the amount of these hormones that are free and active in the bloodstream. Only the unbound (free) hormones can enter cells and exert their effects. Several factors can influence SHBG levels, including age, sex, liver function, use of oral estradiol or testosterone, and the presence of certain medical conditions. Abnormal SHBG levels can indicate various health conditions. High SHBG levels, for example, can be seen in hyperthyroidism, liver disease, HIV infection, and anorexia. High SHBG level can reduce the availability of free testosterone, potentially leading to symptoms of testosterone deficiency. Conversely, low SHBG levels can be associated with hypothyroidism, obesity, polycystic ovary syndrome (PCOS), and use of anabolic steroids, and can result in excessive free testosterone, which might be seen in conditions like PCOS or insulin resistance.

Approximately 60-70% of testosterone binds to SHBG, which binds more tightly and makes this fraction less readily available for tissue uptake. Only about 1-2% of testosterone remains free and biologically active. Free testosterone is rapidly metabolized (T_{1/2} approximately 10 min.) which demonstrates the importance of SHBG binding and dissociation capacity, indicating that SHBG is the major determinant of the free fraction equilibrium. SHBG levels can be measured through a blood test. This measurement is often used in conjunction with tests for total testosterone to calculate the free testosterone level, which is important in diagnosing various hormonal disorders. SHBG is present in much lower concentrations compared to albumin.

The normal range for SHBG can vary depending on age, sex, and the laboratory conducting the test. In general, normal SHBG level in adult men is approximately from 10 to 57 nmol/L, and for women it's approximately 18 to 144 nmol/L in pre-menopausal women and approximately 14 to 103 nmol/L in post-menopausal women. Factors that may affect SHBG levels include:
Age. In childhood and early adolescence, SHBG levels are generally high. As puberty begins, SHBG levels start to decline. During adulthood, SHBG levels tend to stabilize. However, they can be influenced by various factors, including body weight, insulin levels, and overall hormonal balance. Men generally have lower SHBG levels than women. In women, SHBG levels may fluctuate with menstrual cycles, pregnancy, and (oral) contraceptive use. As people age, SHBG levels typically increase. This is particularly evident in men, where SHBG levels rise with age, leading to a reduction in the levels of bioavailable testosterone. This increase in SHBG is one of the factors contributing to the decline in testosterone levels often seen in older men. In women, SHBG levels also increase with age, especially after menopause, as estrogen levels decline. Higher SHBG levels can further reduce the availability of sex hormones, contributing to symptoms of aging such as reduced libido and bone density changes.
SHBG levels are closely linked to hormonal changes that occur during menopause. Menopause marks the end of a woman's reproductive years, typically occurring around the age of 50, and it is characterized by a decline in estrogen and progesterone production. These hormonal shifts have significant effects on SHBG levels. Use of hormone replacement therapies, oral contraceptives, or medications that influence hormones can alter SHBG levels at different life stages.
Medication, such as oral contraceptives, estrogen therapy, and anti-androgens, hormonal therapies.
Health conditions, for example BMI, liver function, hyperthyroidism, obesity, insulin resistance, and polycystic ovary syndrome (PCOS). Higher BMI is often associated with lower SHBG levels. As people age and their body composition changes, SHBG levels can be affected. Since SHBG is produced in the liver, any changes in liver function (for example with age or disease) can impact SHBG levels.

Testosterone in the bloodstream exists in three main fractions:
1. Free Testosterone:
   Percentage: Approximately 1-2% of total testosterone.
   Description: This fraction is not bound to any proteins and is biologically active. It is able to enter cells and exert its effects directly.
2. Albumin-bound Testosterone:
   Percentage: About 30-40% of total testosterone.
   Description: This fraction is weakly bound to albumin, a protein in the blood. It is considered bioavailable because the bond is not strong, allowing testosterone to be readily available for tissue uptake.
3. SHBG-bound Testosterone:
   Percentage: Approximately 60-70% of total testosterone.
   Description: This fraction is strongly bound to SHBG and is generally considered biologically inactive. SHBG-bound testosterone cannot easily enter cells, thus limiting its immediate biological effects.

Free testosterone is the most important fraction for determining androgenic activity, as it represents the amount of hormone available to tissues. The sum of free testosterone and albumin-bound testosterone is bioavailable testosterone, and it is often used in clinical settings to access testosterone levels more accurately, as both fractions can exert biological effects. Total testosterone includes all three fractions, and is commonly measured. It does not always provide a complete picture of hormonal activity without considering SHBG levels.

In addition to “bioavailable testosterone,” the term “bioactive testosterone” is also often used in the context of hormone physiology. Bioactive testosterone refers to the fraction of testosterone that is biologically active, meaning it can bind to androgen receptors in target tissues and elicit a physiological response. Whilst bioavailable testosterone include both free testosterone and albumin-bound testosterone, bioactive testosterone only includes free testosterone, because this is the fraction that can freely enter cells and bind to androgen receptors. Once free testosterone enters a cell, it binds to androgen receptors, leading to various biological effects, such as muscle growth, secondary sexual characteristics, and maintenance of libido. Bioactive testosterone directly reflects the hormone's capability to trigger a biological response within target tissues, and bioavailable testosterone is a more accurate indicator of the testosterone that is accessible to tissues for biological activity, as it includes both free and easily releasable testosterone.

Bioavailable testosterone can be measured by considering both free and albumin-bound testosterone levels. This can be assessed through specific blood tests. Bioactive Testosterone: It is effectively measured by the levels of free testosterone alone, which can be directly tested.

Both bioavailable and bioactive testosterone have important clinical relevance. For example, bioavailable testosterone is important for understanding the overall amount of testosterone that can potentially be used by the body, considering both free and weakly bound forms; and bioactive testosterone is critical for assessing the immediate hormonal activity and effects within the body, as it directly impacts physiological functions.

Albumin levels impact the bioavailability of testosterone. In particular, albumin binds to about 30-40% of the testosterone in the blood. This bound testosterone is considered bioavailable because it is loosely attached to albumin and can easily dissociate to become active. When albumin levels are low, the total amount of albumin-bound testosterone decreases. the proportion of free testosterone (unbound) might increase. However, this does not necessarily mean that more testosterone is available to tissues. Free testosterone can be quickly degraded, and without sufficient albumin, the transport and regulation of testosterone are compromised. This could result in a reduction of bioavailable testosterone. Even if total testosterone levels remain stable, reduced albumin-bound testosterone can lead to symptoms of low testosterone. These include fatigue, decreased libido, erectile dysfunction, mood changes, and muscle weakness.

In the context of the disclosure, albumin levels can also influence the pharmacokinetics and efficacy of the PDE5 inhibitor such as sildenafil. Sildenafil is partially bound to plasma proteins, including albumin. Normally, about 96% of sildenafil in the bloodstream is bound to proteins, with albumin being one of the primary binding proteins. When albumin levels are low, there is less protein available to bind to sildenafil. This can lead to a higher proportion of free (unbound) sildenafil in the blood. Higher levels of free sildenafil may enhance the drug's effects, potentially improving its efficacy in treating erectile dysfunction (or vaginal bloodflow) or pulmonary hypertension. However, a higher concentration of free sildenafil can also increase the risk of side effects, such as headaches, flushing, dizziness, or more severe cardiovascular effects. With less albumin to bind to, free sildenafil may be more rapidly metabolized and cleared from the body, potentially shortening its duration of action. Patients with low albumin might experience more variability in how they respond to sildenafil, with either more pronounced effects or quicker subsidence of its action.

The relationship between SHBG levels and bioavailability of testosterone is more straightforward. Higher SHBG levels lead to high proportion of SHBG-bound testosterone, thereby reducing the amount of testosterone readily available for biological activity.

There are important differences between the usual chronic treatment with modest increased levels of testosterone (by means of patches, gels, pills, etc.) and the treatment according to the disclosure (for example, in the form of sublingual administering of at least 0.5 mg testosterone). Chronic treatment with modest levels of testosterone is accompanied by a slight increase in absolute levels of testosterone, and in the long run (days/weeks), an increase in the free fraction of testosterone. This gradual increase of the free fraction of testosterone is the result of an alteration in a homeo-static state regarding testosterone (metabolism), and is responsible for its positive as well as negative effects. In contrast, in our approach we produce a considerable increase in absolute levels of testosterone (about 15-20 times higher than in the chronic treatment) within and during a (very) short period, and we assume that this increase will produce a transient proportional increase in the free fraction of testosterone. We postulate an SHBG (and to a lesser extent albumin) saturation threshold mechanism. The increase in influx of testosterone into the body will be first bound to SHBG and (to a lesser extent) albumin, before it can produce an increase in the free fraction.

Without wishing to be bound by theory, the dose of testosterone according to the pharmaceutical composition of the disclosure is high enough to saturate the albumin and SHBG (i.e., the concentration of testosterone is high enough to overcome complete binding of testosterone by SHBG or albumin) such that at least a fraction of testosterone not bound by albumin and SHBG is present in the blood (free testosterone).

For this purpose, the pharmaceutical composition of the disclosure comprises no less than 1 mg, for example 1-5 mg, preferably 1-4 mg, testosterone. We have tentatively calculated that the increase of testosterone in the pharmaceutical composition of the disclosure caters for women with an elevated plasma SHBG level, or for example post-menopausal women, with or without combined hormone replacement therapy (HRT), or pre-menopausal women who are taking COC (combined oral contraceptive) medications. It is large enough to “saturate” SHBG in the bloodstream, with sufficient bioavailable testosterone left for biological activity. Thus the pharmaceutical composition of the disclosure will produce a short peak of free testosterone in the patient, and we believe that this short burst in the free fraction of testosterone is responsible for cognitive, affective, and behavioral effects a few hours later. Preferably, the pharmaceutical composition of the disclosure comprises 1 mg, 1.5 mg, 2 mg, 2.5 mg, 3 mg, 3.5 mg, or 4 mg testosterone.

A preferred pharmaceutical composition suitable for the identified patient comprises 1.0 mg testosterone and 100 mg sildenafil. As discussed above, albumin binds (weakly) to both testosterone and sildenafil. Therefore the amounts of testosterone and sildenafil present in the pharmaceutical composition need to be balanced to achieve the desirable synergistical effects. It was found that the combination of 1.0 mg testosterone and 100 mg sildenafil is a promising dose combination for enhanced efficacy for the identified patients.

Optionally, testosterone and the PDE5 inhibitor may present in the pharmaceutical composition in different dosage forms. For example, the PDE5 inhibitor and/or testosterone may be present in a solid dosage form, for example a solid dosage form selected from the group consisting of tablets, capsules, pills, granules, chewables and powders. Testosterone and the PDE5 inhibitor may be of the same or different dosage forms. For example, testosterone may be present in a form suitable for oral or intranasal inhalation and the PDE5 inhibitor may be present in a tablet. Naturally, the pharmaceutical composition of the disclosure does not have to be a single formulation. Rather, it can be a mixture of a composition comprising testosterone and a composition comprising PDE5 inhibitor, as long as the compositions are prescribed or instructed to use together.

Optionally, the combination therapy of the disclosure is a kit comprising a formulation comprising testosterone and a formulation comprising a PDE5 inhibitor. Testosterone is present in the kit in an amount of no less than 1 mg, for example 1-5 mg, preferably 1-4 mg. Each composition preferably further comprises one or more pharmaceutically acceptable excipients selected from the group consisting of solvents, gelling agents, diluents, disintegrants, lubricants, glidants, fillers, binders, surfactants, colorants/pigments, flavorings, sweeteners, plasticizers, moisturizers, antioxidants, water absorbents, coating agents and preservatives. Separate formulations give great flexibilities to medical practitioners when personalizing the required or recommended combination therapy for individual patients. Testosterone and the PDE5 inhibitor such as sildenafil may be formulated in any formulation suitable for use as a combination therapy.

According to a fifth aspect, the disclosure also provides a method for determining a dosage for a patient in the treatment of FSIAD. The method comprises choosing a testosterone dosage of no less than 1.0 mg in combination of a PDE5 inhibitor if:
the patient is post-menopausal; or
the patient is using COCs or receiving HRT; or
the patient has a BMI (Body Mass Index) no greater than a pre-determined threshold, for example 25, 24.5, 24, 23.5, 23, or preferably 22.5; or
the patient has a plasma SHBG (sex hormone-binding globulin) level of no less than pre-determined threshold, for example 80 nmol/L, 90 nmol/L, or preferably 100 nmol/L.

Preferably, the method further comprises choosing sildenafil as the PDE5 inhibitor and/or choosing a PDE5 inhibitor dosage of no less than 25 mg, no less than 50 mg, for example 25 mg, 50 mg, 75 mg or 100 mg.

Optionally, the method comprises choosing a pharmaceutical composition comprising 1.0 mg testosterone and 100 mg sildenafil for the patient.

According to a further aspect, the disclosure also provides a method of determining a dosage regime of a pharmaceutical composition for a patient in the treatment of FSIAD. The pharmaceutical composition comprises sildenafil and testosterone, and the method comprises:
a) collecting the following information of the patient:
   i) the patient's menopausal status;
   ii) the patient's BMI;
   iii) if the patient is using COCs or receiving HRT;
   iv) the plasma SHBG level of the patient; and
   v) the plasma albumin level of the patient,
b) comparing the information collected to a pre-determined schedule; and
c) choosing a dosage regime of sildenafil and/or testosterone for the patient based on the comparison.

According to a further aspect, the disclosure provides a method of enhancing female sexual desire in the treatment of FSIAD of a patient. The method comprises:
a) collecting information on the following impactor factors of the patient:
   i) the patient's menopausal status;
   ii) the patient's BMI;
   iii) if the patient using COCs or receiving HRT;
   iv) the plasma SHBG level of the patient; and
   v) the plasma albumin level of the patient
b) comparing the information collected to a pre-determined schedule; and
c) administering a pharmaceutical composition to the patient, wherein the pharmaceutical composition comprises sildenafil and testosterone.

The pre-determined schedule may include rules such that the patient receives a pharmaceutical composition of the disclosure, preferably a pharmaceutical composition of the disclosure comprising no less than 1 mg, for example 1.0 mg, 1.5 mg, 2.0 mg, 2.5 mg, 3.0 mg, 3.5 mg, or 4.0 mg, testosterone, preferably in an immediate release dosage form such as oral, sublingual, or intranasal, if:
the patient is post-menopausal; or
the patient is using COCs or receiving HRT; or
the patient has a BMI (Body Mass Index) no greater than a pre-determined threshold, for example 25, 24.5, 24, 23.5, 23, or preferably 22.5; or
the patient has a plasma SHBG (sex hormone-binding globulin) level of no less than pre-determined threshold, for example 80 nmol/L, 90 nmol/L, or preferably 100 nmol/L; or
the patient also has a plasma albumin level no greater than a pre-determined threshold, for example 45 g/L, 40 g/L, or preferably 37 g/L.

Preferably, the pre-determined schedule further includes rules such that the patient receives a pharmaceutical composition comprising 0.5 mg testosterone in combination with less than 100 mg, for example 75 mg or 50 mg or 25 mg, PDE5 inhibitor, such as sildenafil, if the patient does not meet any of the criteria above.

Preferably, the method further comprises treating the patient for a pre-determined period of time according to the schedule, evaluating the efficacy and side effects of the treatment, and adjusting the dosage of testosterone and the PDE5 inhibitor based on the evaluation.

For example, if, according to the evaluation, the efficacy of the treatment is not sufficient, the dosage of testosterone can be adjusted. If testosterone dosage in the treatment was 0.5 mg, it can be increased to, for example 1.0 mg or 1.5 mg, administered in the form of one or more compositions (for example two compositions comprising 0.5 mg and 1.0 mg testosterone, respectively, for the total dosage of 1.5 mg). If testosterone dosage in the treatment was 1.0 mg, it can be adjusted to 2.0 mg. The 2.0 mg testosterone can be administered by means of the pharmaceutical composition of the disclosure in combination with a further pharmaceutical composition comprising 1.0 mg testosterone, for example an oral 1.0 mg testosterone composition, or a 1.0 mg testosterone composition in a solid dosage form such as a tablet, a capsule, or in a dosage form suitable for oral or intranasal inhalation.

Alternatively, if the side effects of the treatment are too severe, the dosage of the PDE5 inhibitor can be adjusted. If PDE5 inhibitor (such as sildenafil) dosage in the treatment was 50 mg, it can be reduced to, for example 25 mg. If PDE5 inhibitor (such as sildenafil) dosage in the treatment was 100 mg, it can be reduced, for example to 75 mg or 50 mg. The dosage of testosterone can be adjusted at the same time.

In a non-limiting embodiment of the disclosure, a patient is initially treated with a pharmaceutical composition comprising 1.0 mg testosterone and 100 mg sildenafil, for example a pharmaceutical composition of the disclosure, for a period of time such as 8 weeks, and is subsequently treated with a pharmaceutical composition comprising 1.0 mg testosterone and 100 mg sildenafil, for example a pharmaceutical composition of the disclosure, in combination with a further pharmaceutical composition comprising 1.0 mg testosterone, when the initial treatment does not provide sufficient efficacy.

In a non-limiting embodiment of the disclosure, a patient is initially treated with a pharmaceutical composition comprising 1.0 mg testosterone and 100 mg sildenafil, for example a pharmaceutical composition of the disclosure, for a period of time such as 8 weeks, and is subsequently treated with a pharmaceutical composition comprising 0.5 mg testosterone and 50 mg sildenafil, optionally in combination with a further pharmaceutical composition comprising 1.0 mg testosterone, when the initial treatment induces too severe side effects.

In a non-limiting embodiment of the disclosure, a patient is initially treated with a pharmaceutical composition comprising 0.5 mg testosterone and 50 mg sildenafil, for a period of time such as 8 weeks, and is subsequently treated with a pharmaceutical composition comprising 1.0 mg testosterone and 100 mg sildenafil, for example a pharmaceutical composition of the disclosure, or a pharmaceutical composition comprising 0.5 mg testosterone and 50 mg sildenafil in combination with a further pharmaceutical composition comprising 1.0 mg testosterone, when the initial treatment does not provide sufficient efficacy.

In a non-limiting embodiment of the disclosure, a patient is initially treated with a pharmaceutical composition comprising 0.5 mg testosterone and 50 mg sildenafil, for a period of time such as 8 weeks, and is subsequently treated with a pharmaceutical composition comprising 0.5 mg testosterone and 25 mg sildenafil, when the initial treatment induces too severe side effects.

The further pharmaceutical composition comprising 1.0 mg testosterone may be an oral 1.0 mg testosterone composition, or a 1.0 mg testosterone composition in a solid dosage form such as a tablet, a capsule, or in a dosage form suitable for oral or intranasal inhalation.

The efficacy and side effects of the treatment may be evaluated by any methods suitable for use in the art. The skilled person is familiar with tools and protocols for such evaluations, based on the common general knowledge. A non-limiting example of evaluating the efficacy and side effects of a treatment for FSIAD is described in more detail in Example 1.

According to a further aspect, the disclosure provides testosterone or a functional analogue or derivative thereof in an amount equivalent to at least 1.0 mg testosterone for use in combination with a PDE5 inhibitor, preferably sildenafil, in enhancing female sexual desire in the treatment of FSIAD of a patient, and the patient:
is post-menopausal or
the patient is using COCs or receiving HRT; or
has a BMI (Body Mass Index) no greater than 22,5; or
has a plasma SHBG (sex hormone-binding globulin) level of no less than 100 nmol/L.

Preferably, the testosterone or the functional analogue or derivative thereof is used in combination with sildenafil in an amount of no less than 25 mg, no less than 50 mg for example 50 mg, 75 mg, for example 100 mg.

According to a further aspect, the disclosure provides sildenafil in an amount of no less than 25 mg, no less than 50 mg, preferably no less than 25 mg, no less than 50 mg, for example 50 mg, 75 mg, or 100 mg, for use in combination with testosterone or a functional analogue or derivative thereof in enhancing female sexual desire in the treatment of FSIAD of a patient, and the patient has a BMI no greater than 22.5; or a plasma albumin level of no greater than 37 g/L. Preferably, the testosterone or the functional analogue or derivative thereof is used in an amount equivalent to no less than 1.0 mg, for example 1-5 mg, preferably 1-4 mg, testosterone.

Any feature that has been described above in relation to any one aspect or embodiment of the disclosure is also disclosed hereby in relation to all other aspects and embodiments. Likewise, all combinations of two or more of the individual features or elements described above may be present in any aspect or embodiment. For brevity, all possible features and combinations have not been recited in relation to all aspects and embodiments, but they are expressly contemplated and hereby disclosed.

### BRIEF DESCRIPTION OF THE DRAWINGS

FIG. 1 is a diagram showing the study plan of Example 2.

FIGS. 2A to 2G are diagrams showing numbers (and percentages) of participants showing improvements in each category in Example 3.

### DETAILED DESCRIPTION

### Examples

The disclosure will be explained in more detail in the following, non-limiting examples.

### Example 1

Study population: European premenopausal women aged between 21 to 70 years with acquired FSIAD diagnosed by a trained professional in accordance with the Diagnostic and Statistical Manual of Mental Disorders-5 (DSM-5).

Interventions: Participants will receive a drug product, which is a fixed-dose combination tablet consisting of an inner core containing sildenafil citrate of one of the two different dose strengths (resp. 25 & 50 mg) with an outer delayed immediate release coating and an additional outer film coating containing testosterone of one of the two different dose strengths (resp. 0.25 & 0.5 mg), or a placebo. The drug product is a 9-mm, round, biconvex, white, menthol-flavored tablet for sublingual administration. Sildenafil is released immediately after the delay of approximately 2.5 hours after the administration. The placebo tablets were identical in appearance and flavor to the drug product.

Study design: A double-blind, randomized, placebo-controlled 6-month study.

### Experimental Part

The experimental will be preceded by a screening visit. Participants who had been diagnosed with any psychiatric disorder other than FSIAD were excluded. Those scheduled for any other treatment for female sexual dysfunction during the study period also were excluded. Potential participants were excluded if they were using oral contraceptives containing anti-androgens, estrogen more than 50 μg, potent cytochrome P450 3A4 inhibitors or inducers, nitrates, monoamine oxidase inhibitors, antidepressants, and/or T compounds. Potential participants with cardiovascular conditions, gynecologic conditions were also excluded. Women with clinically relevant endocrine disease, neurologic disease, severe or acute liver disease, or a history of severe hepatic impairment were excluded. Participants were excluded if they had free and/or total T levels that were beyond the upper limit of the central laboratory's reference range. Eligible participants were assigned to 1 of the 2 FSIAD subgroups based on a demarcation formula.

After a baseline period of 4 weeks, participants underwent an 8-week, single-blinded, placebo run-in (PRI) period, which was intended to stabilize placebo responses before active treatment. The PRI period was followed by an 8-week, double-blinded ATP. Those participants were randomly assigned to one of the following treatment arms: i) placebo, ii) S 50 mg, iii) T 0.5 mg, iv) T 0.25 mg+S 25 mg, v) T 0.25 mg+S 50 mg, vi) T 0.5 mg+S 25 mg, or vii) T 0.5 mg+S 50 mg. Sample sizes of randomized population are shown in Table 1 below:

TABLE 1
Sample sizes of randomized population

| | | 0.25 mg T/ | 0.25 mg T/ | 0.5 mg T/ | 0.5 mg T/ | | | |
|---|---|---|---|---|---|---|---|---|
| | Placebo | 25 mg S | 50 mg S | 25 mg S | 50 mg S | 0.5 mg T | 50 mg S | Total |
| Randomized | 33 | 32 | 32 | 32 | 32 | 32 | 32 | 225 |
| ITT | 26 | 27 | 29 | 27 | 27 | 30 | 30 | 196 |

Participants were instructed to keep the tablet under their tongues for 90 seconds and then to swallow it whole, without chewing it or otherwise disrupting the dosage form. They were permitted to take the tablet with a little water. The participants were instructed to engage in sexual activity 3-6 hours after ingestion The dosing instructions were the same for all regimes. 28 doses were provided in the 8-week treatment period. The participants were asked to endeavor to take a minimum of 1 dose per week. They were informed that they could take the remaining doses as required (ie, on demand) throughout the 8-week treatment period. The minimum period between individual doses was 2 days (ie, on alternate days).

The participants were interviewed every 4 weeks, at which time they also completed questionnaires designed to assess sexual function and mood. In addition, their vital signs were measured, any signs of hyperandrogenism were evaluated, a pregnancy test was administered, and any adverse events were monitored. A psychological interview was conducted at weeks 8 and 16 to evaluate each 8-week treatment period, at which time blood samples were collected to assess each participant's chemistry, hematology, lipid and hormones.

Efficacy of the drug product was measured using the validated Sexual Event Diary (SED) and Weekly Diary (WD). Participants are required to complete the SED within a period of 24 hours after each sexual event. This information was used to assess each participant's satisfaction and sexual functioning during that event. In the Diary, they were asked to answer questions relating to the level of sexual desire, pleasure, bodily arousal and subjective arousal according to 5-point Likert scale items).

### Results

Baseline values for diary questions related to sexual desire and distress/arousal for the placebo group, the treatment groups and the control groups are shown in Table 2 below.

TABLE 2
Baseline values for diary questions related to Desire and Distress/Arousal

| | | Q6: | Q7: | Q8: | Q9 | Q13 | Desire | Inhib. |
|---|---|---|---|---|---|---|---|---|
| | Q5: | Let | Sexual | Bodily | Sexually | Distract. | Domain | Domain |
| | Pleasurable | Go | Desire | Arousal | Aroused | Thoughts | (Q7/Q9) | (Q6/Q13) |
| A. Placebo | 2.81 | 2.78 | 2.32 | 3.06 | 2.39 | 2.40 | 2.36 | 2.69 |
| B. 0.25 mg T + 25 mg S | 3.12 | 3.00 | 2.47 | 3.56 | 2.75 | 2.45 | 2.61 | 2.77 |
| C. 0.25 mg T + 50 mg S | 2.49 | 2.41 | 2.28 | 3.14 | 2.29 | 2.36 | 2.28 | 2.52 |
| D. 0.5 mg T + 25 mg S | 2.76 | 2.67 | 2.24 | 2.93 | 2.28 | 2.19 | 2.26 | 2.74 |
| E. 0.5 mg T + 50 mg S | 2.52 | 2.43 | 2.45 | 3.07 | 2.25 | 2.41 | 2.35 | 2.51 |
| F. 0.5 mg T | 2.78 | 2.68 | 2.16 | 3.08 | 2.42 | 2.36 | 2.29 | 2.66 |
| G. 50 mg S | 2.68 | 2.59 | 2.28 | 3.21 | 2.54 | 2.57 | 2.41 | 2.51 |
| Overall | 2.73 | 2.64 | 2.31 | 3.15 | 2.42 | 2.40 | 2.36 | 2.62 |

Changes from baseline following ANCOVA corrected for baseline for diary questions for all groups are shown in Table 3 below.

TABLE 3
Changes from baseline following ANCOVA corrected for
baseline (F-test p-value 0.0649-0.15)

| | | Q8: | Q9: | Q7/Q9 |
|---|---|---|---|---|
| | Q5: | Bodily | Sexually | Desire |
| | Pleasurable | Arousal | aroused | Domain |
| | F-test: | F-test: | F-test: | F: test: |
| | 0.0699 | 0.0649 | 0.0961 | 0.1226 |
| A. Placebo | 0.61 | 0.34 | 0.74 | 0.70 |
| B. 0.25 mg T + | 0.36 | 0.31 | 0.51 | 0.60 |
| 25 mg S | | | | |
| C. 0.25 mg T + | 0.60 | 0.55 | 0.68 | 0.74 |
| 50 mg S | | | | |
| D. 0.5 mg T + | 0.79 | 0.93 | 0.97 | 0.95 |
| 25 mg S | | | | |
| E. 0.5 mg T + | 1.19 | 0.61 | 1.29 | 1.28 |
| 50 mg S | | | | |
| F. 0.5 mg T | 0.45 | 0.32 | 0.48 | 0.55 |
| G. 50 mg S | 0.64 | 0.22 | 0.72 | 0.71 |

Participants in groups B, C and D reported improvements on sexual desire (as well as bodily arousal) when compared to the relevant baselines. The changes for the desire domain is significantly higher in treatment group E with 0.5 mg Testosterone and 50 mg sildenafil, although the effect in treatment group D with 0.5 mg Testosterone and 25 mg sildenafil is also noticeable.

Further analysis was performed for pairwise comparisons of 6 lower dose groups with the highest does group D, and the results are shown in Table 4 below.

TABLE 4
ANCOVA results of diary questions related to Desire and Arousal:

| | | p-values of pairwise comparison of 6 lower dose groups with | | | | | |
|---|---|---|---|---|---|---|---|
| | ANCOVA | highest dose group E. 0.5 mg Testosterone and 50 mg Sildenafil | | | | | |
| | Overall F- | | B. | C. | D. | | |
| | test | A. | 0.25 mg T/ | 0.25 mg T/ | 0.5 mg T/ | F. | G. |
| | p-values | Placebo | 25 mg S | 50 mg S | 25 mg S | 0.5 mg T | 50 mg S |
| Q5: Pleasurable | 0.0699 | X | X | X | | X | X |
| Q6: Let Go | 0.4032 | | | | | | |
| Q7: Sexual | 0.2354 | X | X | | | X | X |
| Desire | | | | | | | |
| Q8: Bodily | 0.0649 | Z | Z | | | Z | Z |
| Arousal | | | | | | | |
| Q9: Sexually | 0.0961 | | X | X | | X | X |
| Aroused | | | | | | | |
| Q13: Distracting | 0.6468 | | | | | | |
| Thoughts | | | | | | | |
| Desire Domain | 0.1226 | X | X | X | | X | X |
| (Q7/Q9) | | | | | | | |
| Inhibition | 0.4116 | X | | | | | |
| Domain | | | | | | | |
| (Q6/Q13) | | | | | | | |

X: p-value for pairwise treatment comparison (no correction for multiplicity) with 0.5 mg/50 mg dose group is <0.05
Z: p-value for pairwise treatment comparison (no correction for multiplicity) with 0.5 mg/25 mg dose group is <0.05

The results in Table 3 show that for several parameters lower dose-groups have a p<0.05 when directly compared with the highest dose group (X). Dose group 0.5 mg Testosterone/25 mg Sildenafil shows no difference with the highest dose group (X), and for Q8 this dose group shows an effect compared to the lower dose groups that is not seen comparing these with the highest dose group (Z).

Although none of the F-tests results in Table 3 reached statistical significance (p<0.05), there are several F-tests with p-values in the range 0.05-0.15, for which is was explored whether these were an indication of an underlying dose-response of the testosterone or sildenafil components.

The dose-response of testosterone at sildenafil 50 mg dose is shown in Table 5 below.

TABLE 5
Dose-response Testosterone at Sildenafil = 50 mg, based on
ANCOVA corrected for baseline for questions with
overall 7 treatments F-test p-value 0.0649-0.15

| | | Q8 | Q9: | Q7/Q9 |
|---|---|---|---|---|
| | Q5: | Bodily | Sexually | Desire |
| Treatment | Pleasurable | Arousal | aroused | Domain |
| G. 50 mg Sil | 0.64 X | 0.22 | 0.72 X | 0.71 X |
| C. 0.25 mg Test + 50 mg Sil | 0.60 X | 0.55 | 0.68 X | 0.74 X |
| E. 0.5 mg Test + 50 mg Sil | 1.19 | 0.61 | 1.29 | 1.28 |

X: p-value for pairwise treatment comparison (no correction for multiplicity) with 0.5 mg/50 mg dose group is < 0.05

The dose-response of sildenafil at testosterone 0.5 mg dose is shown in Table 6 below.

TABLE 6
Dose-Response Sildenafil at Testosterone = 0.5 mg, based on
ANCOVA corrected for baseline for questions with
overall 7 treatments F-test p-value 0.0649-0.15

| | | Q8: | Q9: | Q7/Q9 |
|---|---|---|---|---|
| | Q5: | Bodily | Sexually | Desire |
| Treatment | Pleasurable | Arousal | aroused | Domain |
| F. 0.5 mg Test | 0.45 X | 0.32 Z | 0.48 X | 0.55 X |
| D. 0.5 mg Test + 25 mg Sil | 0.79 | 0.93 | 0.97 | 0.95 |
| E. 0.5 mg Test + 50 mg Sil | 1.19 | 0.61 | 1.29 | 1.28 |

X: p-value for pairwise treatment comparison (no correction for multiplicity) with 0.5 mg/50 mg dose group is < 0.

The dose-response of placebo-mid dose-high dose at sildenafil 50 mg dose is shown in Table 7 below.

TABLE 7
Dose-Response Placebo-Mid-High, based on
ANCOVA corrected for baseline for questions with
overall 7 treatments F-test p-value 0.0649-0.15

| | | Q8: | Q9: | Q7/Q9 |
|---|---|---|---|---|
| | Q5: | Bodily | Sexually | Desire |
| Treatment | Pleasurable | Arousal | aroused | Domain |
| A. Placebo | 0.61 X | 0.34 | 0.74 | 0.70 X |
| B. 0.25 mg Test + 25 mg Sil | 0.36 X | 0.31 | 0.51 X | 0.60 X |
| E. 0.5 mg Test + 50 mg Sil | 1.19 | 0.61 | 1.29 | 1.28 |

X: p-value for pairwise treatment comparison (no correction for multiplicity) with 0.5 mg/50 mg dose group is < 0.

For Q5 which measures the level of sex pleasure, as well as for the desire domain (Q7/Q9), the highest dose 0.5 mg Testosterone and 50 mg sildenafil (Treatment Group E) shows a higher response than the lower doses or the two components separately. A dose response of sildenafil is seen at Testosterone 0.5 mg (Table 6). The other combinations do not show a clear dose-response: At Sildenfal 50 mg (Table 5), placebo Testosterone and Testosterone 25 mg show a similar response. Mid-dose result in Table 7 shows no dose-response.

For Q8 which relates to bodily arousal, Tables 3 to 7 show that treatment group D shows a higher response than lower doses and the 2 components separately. The lower response of the highest dose group E, does not support dose-response of question.

Overall, the results of the experiment demonstrate that the combination of immediate release testosterone and delayed immediate release sildenafil outperformed the placebo and monotherapies over an 8-week period for sexual desire and sexual arousal for women with FSIAD. Higher dose of testosterone (0.5 mg vs 0.25 mg) appears to enhance the performance of the drug combination. For sildenafil, improvements start to show with the lower dose (25 mg, when combined with 0.25 mg testosterone). The highest-dose combination of testosterone and sildenafil produced statistically significant increase, and appears to be a very promising treatment option for enhancing female sexual desire in the treatment of FSIAD.

### Example 2

Study Rationale: In sex research, it might be desirable to measure physiological and subjective sexual responses. The subjective responses are usually measured with validated questionnaires while physiological methods request physical measurements.

A relatively straightforward and common method to induce sexual excitation in the laboratory is by exposing subjects to validated erotic film fragments. Relatively nonintrusive psychological manipulations can shift the delicate balance between excitatory and inhibitory factors influencing the sexual response. This implies that systematic factors present in the institutional sex laboratory setting-such as presence of an experimenter in the other room, or the unfamiliar artificial situation-might influence the sexual response in an unknown manner, and thus bias the results.

Questionnaires are often used in the laboratory to assess subjective arousal. Vaginal photoplethysmography (VPP) has been widely used to measure the genital response to erotic stimuli (e.g., exposure to erotic film excerpts). This measurement method has been validated as specific to erotic stimuli. The use of the vaginal photoplethysmography over the past three decades has enriched our understanding of the factors that influence and are implicated in female sexual arousal. Because of the absence of an absolute metric in the VPP, within-subjects protocols are necessary wherein the subject is exposed to neutral and subsequently erotic stimuli, and responses to the two conditions are compared. Two signals are derived from the VPP: one that reflects a general pooling of blood in the vaginal tissue (vaginal blood volume) and one that reflects moment-to-moment changes in blood flow that occur with each heartbeat, with larger amplitudes reflecting higher levels of vasocongestion (vaginal pulse amplitude; VPA). Because of its sensitivity and specificity, VPA is the preferred measure when using the VPP. VPP remains a popular instrument for the study of female psychophysiology because of practical advantages such as its ability to be inserted by the research subject, its ease of disinfection and handling, its relative durability with repeated use, the low cost of its laboratory hardware, and the fact that it is relatively unobtrusive once inserted. The VPP has been employed in a wide array of research designs and in response to varied research questions pertaining to genital arousal. For example, it has been used to study gender differences, effects of menopause, female sexual dysfunction, sexual orientation, and in pharmacotherapy outcome trials.

Clitoral photoplethysmography for the measurement of clitoral blood volume (CBV) has also been used. Main effects of the film and the interaction between the film and epoch were stronger for the clitoral than for the vaginal device. In addition, Clitoral Pulse Amplitude (CPA) followed more closely changes in intensity of sexual films than VPA.

Experiments in men mostly show high concordance between their physiological and subjective arousal, and as a result these subjective measures are more valid in men. In women this relationship is less clear, frequently showing discordance between measures of genital and subjective arousal [investigated sexual responses in women suffering from hypoactive sexual desire disorder (HSDD) and in a control group of sexually functional women (healthy controls). Women would (i) show more attention for subliminally presented erotic stimuli (as measured by a masked version of the Emotional Stroop Task), (ii) show more genital sexual arousal (as measured by vaginal pulse amplitude [VPA] and clitoral blood volume (CBV), and (iii) experience stronger sexual arousal (as measured by the Sexual Arousal Response Questionnaire.

Studies show that in healthy controls, clitoral and subjective laboratory measures of sexual arousal show stronger increases to erotic stimuli in the home environment than in the environment of the institutional laboratory. In the institutional laboratory, women with HSDD and healthy controls did not differ in their genital and subjective response to erotic stimuli. The genital arousal to erotic stimuli of women with HSDD and of healthy controls only differed from each other in the ambulatory laboratory setting.

The literature on efficacy of sildenafil in women with sexual dysfunction has been mixed with reports of (1) no effect in two large diagnostically heterogeneous groups of estrogenized and non-estrogenized women with sexual dysfunction that included impairment of arousal, (2) marginally increased subjective sexual arousal and vaginal vasocongestion in women with spinal cord injury and (3) significantly increased self-reported sexual arousal in premenopausal women with acquired genital female sexual arousal disorder. Overall, the data suggest that sildenafil was ineffective at restoring orgasmic capacity or affecting subjective sexual arousal. However, when the vaginal pulse amplitude of low responders were compared with women who did show a robust increase in vaginal pulse amplitude with the erotic film (vaginal pulse amplitude high responders), differential responses to sildenafil emerged. Specifically, vaginal pulse amplitude of low responders showed significantly reduced latency to orgasm, significantly higher perception of genital arousal and significantly higher levels of overall subjective sexual arousal with sildenafil. Overall, these data suggest that sildenafil may be useful for a subgroup of postmenopausal women with acquired genital female sexual arousal disorder and that use of the vaginal photoplethysmography might be helpful in their delineation.

This study will aim to show the objective effect of the medication in premenopausal females with FSIAD in home setting.

### Primary Objective & Endpoint:

The primary objective of Example 2 is to evaluate the efficacy of testosterone & sildenafil combination therapy relative to placebo in premenopausal women with acquired generalized FSIAD.

The primary endpoint is the mean change from baseline to peak vaginal pulse amplitude (VPA), over (up till) 4 measurements in 2 weeks of treatment, as estimated by vaginal photoplethysmography (VPP) during visual sexual stimulation (VSS) with different types of erotic films. Baseline is measured during neutral films.

### Secondary Objective & Endpoint:

The secondary objective of Example 2 is to evaluate the efficacy of testosterone & sildenafil combination therapy relative to placebo in premenopausal women with acquired generalized FSIAD, by assessing responses to various questionnaires measuring sexual function, satisfaction and distress associated with sexual function.

The secondary endpoint is the mean change from baseline over (up till) 4 measurements in 2 weeks of treatment, in the following measures, after each session of visual sexual stimulation (VSS) with different types of erotic films, and baseline measured during neutral films:
Sexual desire assessed by Female Sexual Function Index Desire Domain (FSFI-D) (items 1 and 2);
Sexual distress assessed by Female Sexual Distress Scale-Desire/Arousal/Orgasm (FSDS-DAO) score for item 13;
Elements of Desire Questionnaire (EDQ) ‘event-based’*
Sexual distress assessed by FSDS-DAO total score;
Sexual arousal assessed by FSFI Arousal Domain (FSFI-A) (items 3-6);
Sexual arousal assessed by Sexual Arousal Response Questionnaire;
Patient-reported impression of improvement assessed by the Patient Global Impression of Improvement (PGI-I);
Sexual function assessed by FSFI total score;
Frequency of (serious) adverse events ((S)AEs).
for EDQ ‘event-based’ the daily version of the EDQ will be used within 24 hours of an event, whereby an event is defined as a measurement of VPA after the actual dosing of the study drug.

### Safety Objective & Endpoint:

The safety objective of Example 2 is to evaluate the safety of testosterone & sildenafil combination therapy relative to placebo.

The safety endpoint is frequency of (serious) adverse events.

### Study Population:

24 healthy premenopausal women at least 18 years of age.

### Inclusion Criteria:

1. Healthy premenopausal woman, at least 18 years of age and able to provide written informed consent (menopausal status based on the Stages of Reproductive Aging Workshop criteria)
2. A clinical diagnosis of acquired, generalized Female Sexual Interest & Arousal Disorder (FSIAD; DSM-5 302.72 (F52.22), as assessed by a trained professional. Subjects should have presence of at least 2 symptoms from the list below:
   Absent/reduced interest in sexual activity
   Absent/reduced sexual/erotic thoughts or fantasies
   No/reduced initiation of sexual activity, and typically unreceptive to a partner's attempts to initiate
   Absent/reduced sexual excitement/pleasure during sexual activity in almost all or all (approx. 75-100%) sexual encounters
   Absent/reduced sexual interest/arousal in response to any internal or external sexual/erotic cues (e.g., written, verbal, visual)
   Absent/reduced genital or nongenital sensations during sexual activity in almost all or all (approximately 75-100%) sexual encounters

At least two of the symptoms present must be hypoactive sexual desire symptoms (i.e., at least two symptoms should be present from the four symptoms numbered 1-4) and reduced/absent arousal.

Symptoms should be persisting or recurrent for a minimum duration of approximately 6 months and should cause marked distress in the individual.

Symptoms are not better explained by a nonsexual mental disorder or as a consequence of severe relationship distress (e.g., partner violence) or other significant stressors and is not attributable to the effects of a substance/medication or another medical condition.
3. Per the subject, previously experienced normal sexual function.
4. Motivated to engage in sexual activity and has the wish to solve their FSIAD.
5. Currently in a stable (>6 months) relationship with a partner of any gender.
6. Non-pregnant and non-breastfeeding. Subjects should have a negative urine (or serum) pregnancy test at screening regardless of childbearing potential. Women of childbearing potential (WOCBP) must comply with using highly effective methods of contraception as defined in Section 6.2.2. See also Section 6.2.2 for acceptable forms of contraception for a male partner. Women of non-childbearing potential may be included without the use of adequate birth control, provided they meet all eligibility criteria.
7. Has all of the following patient reported outcome (PRO) scores at Day 1
8. Either FSFI total score of <=26 or FSFI-D score of <=5
9. FSDS-DAO total score of >18 (range 0 to 60)
10. Normal mammography at screening. Mammography at screening is only to be performed in the absence of a documented normal mammography result obtained within 12 months of screening.
11. Normal gynecological exam at screening
12. Absence of bacterial vaginosis, Candida infection, Chlamydia and gonococcus infection.
13. A clinically acceptable Pap test defined as normal (<2) at screening. A Pap test at screening is only to be performed in the absence of a documented normal result obtained within 12 months of screening.
14. Willing and able to complete all study assessments, follow study instructions and attend all study visits as required, in the opinion of the investigator.
15. A signed Informed Consent Form.

### Exclusion Criteria:

1. Presence of any significant disease/disorder which is unstable or uncontrolled despite treatment, and which in the opinion of the investigator (and if deemed necessary with consultation of the medical monitor), may either put the subject at risk because of participation in the study, may influence the results of the study, or the subject's ability to participate in the study. This includes but is not limited to a subject who:
   has sexual dysfunction other than FSIAD, causing low sexual desire as judged by the investigator
   has unexplained gynecological complaints
   has a genital-pelvic pain/penetration disorder according to DSM-5
   has untreated endocrine disease (e.g., hypopituitarism, hypothyroidism, diabetes mellitus)
   has any clinical signs or symptoms of hyperandrogenism
   has any underlying condition that would preclude sexual activity
   has any current and clinically significant neurological disease
   has any clinically significant abnormality being a contraindication for testosterone and/or sildenafil use
   has a substance abuse disorder
2. History of myocardial infarction (MI), stroke, transient ischemic attack (TIA), or life-threatening arrhythmia within 6 months prior to screening.
3. Current cardiac failure or coronary artery disease causing unstable angina.
4. Uncontrolled hypertension (>140/90 mm Hg) defined as:

Two sequential assessments (approximately 4 minutes apart and no more than 15 minutes apart, after being seated for at least 5 minutes prior to first reading) at levels above 140 mm Hg systolic blood pressure or 90 mm Hg diastolic blood pressure. Subjects who meet either of these criteria at 2 separate visits at least 24 hours apart will be excluded.
5. Treatment for hypertension that has been changed at least once in the 4 weeks before screening.
6. Systolic blood pressure <90 mmHg and/or diastolic blood pressure <50 mmHg.
7. History of loss of vision because of damage to the optic nerve (such as non-arteritic anterior ischaemic optic neuropathy (NAION)).
8. Inherited eye disease (such as retinitis pigmentosa).
9. Intention to become pregnant during the study.
10. Lactating or delivery in the 6 months prior to screening.
11. Acute or chronic hepatitis.
12. Known severe liver and/or renal insufficiency (creatinin clearance of <30 ml/min).
13. History of hormone dependent malignancy (including all types of breast cancer).
14. Active or chronic clinically significant sexually transmitted disease within 6 months before screening.
15. Active moderate to severe vaginitis within 6 months before screening.
16. History of, diagnosed with, and/or is taking or has received treatment for psychosis, bipolar disorder, or depression within the 6 months prior to screening. Note: history of depression that is no longer present is not exclusionary.
17. History of suicide attempt or at increased risk for suicide according to the Beck Scale for Suicidal Ideation. Any subject with a score >0 on questions 1 to 5 and/or question 20 is not eligible and should be referred to their physician as appropriate.
18. Has had a bilateral oophorectomy (or any other procedure that affects menses cycles, eg. endometrial ablation).
19. Has had a hysterectomy AND meets one or more of the following:
   Age was >50 at screening
   Was menopausal by Stages of Reproductive Aging Workshop menstrual cycle criteria before the hysterectomy.
20. FSH was >40 mIU/ml at screening
21. Estradiol was <20 pg/ml at screening
22. Is receiving any treatment to enhance sexual function (eg. pharmacologic, psychotherapy, physical therapy, herbal treatments, dietary supplements) at the time of screening, or plans to take this treatment during the study period.
23. History of (childhood) sexual trauma or abuse that, in the opinion of the investigator, could have negative psychological effects.
24. Current use of:
   any combined hormonal contraceptive containing more than 50 μg/day ethinyl-estradiol or an equivalent of other estrogen,
   strong CYP3A4 inhibitors (ritonavir and erythromycin),
   selective serotonin reuptake inhibitors (SSRIs), if not used in stable regimen and if not taken because of mood symptoms related to the sexual disorder
   serotonin-norepinephrine reuptake inhibitors (SNRIs), if not used in stable regimen and if not taken because of mood symptoms related to the sexual disorder
   tricyclic antidepressants (TCAs) or other antidepressants, if not used in stable regimen and if not taken because of mood symptoms related to the sexual disorder
   monoamine oxidase (MAO) inhibitors
   nitrates or nitric oxide donor compounds
   testosterone and/or phosphodiesterase inhibitor therapy.
25. Unacceptable compliance with completing the e-diary, i.e., during the screening period the subject did not touch the e-diary during two periods of seven consecutive days.
26. Use of any investigational drug/device within 30 days or 5 half-lives of Day 1, whichever is longer, or plans to participate in another study of a drug or device during the study period.

Rescreening of screen failures is allowed if either of the following is the sole reason: 1) blood pressure criteria are out of range, 2) an unexplained laboratory test value is out of range, 3) e-diary compliance that is not acceptable but is expected to reach an acceptable level of compliance based on re-instructing the subject.

Interventions: Participants will receive a drug product, which is a fixed dose combination tablet that contains an inner core containing sildenafil citrate with an outer delayed immediate release coating and an additional outer film coating containing testosterone.

Two dose strengths will be evaluated:
Dose strength 1: 0.5 mg/50 mg (0.5 mg testosterone and 50 mg sildenafil, administered via 1 tablet (Table A) of 0.5 mg testosterone/50 mg sildenafil and 1 placebo tablet)
Dose Strength 1: 1 mg/100 mg (1 mg testosterone and 100 mg sildenafil administered via 2 tablets of Tablet A 0.5 mg/50 mg)

When dosing subjects need to take 2 tablets, the two tablets together should be held sublingually for 90 seconds (release of testosterone), after which the tablets are swallowed as a whole, without chewing or otherwise disrupting the dosage form.

Dosing should occur 3-6 hour prior to the VPP measurement.

Detailed instructions on the dosing procedure will be provided via the instructions for use for the subject.

Placebo tablets, with exactly the same appearance, odor and flavor as Tablet A tablets.

When dosing subjects need to take 2 tablets. The two tablets together should be held sublingually for 90 seconds, after which the tablets swallowed as a whole, without chewing or otherwise disrupting the dosage form.

Dosing should occur 3-6 hour prior to VPP measurement.

Detailed instructions on the dosing procedure will be provided via the instructions for use for the subject.

Study design: A 6-month double-blind, randomized, placebo-controlled, cross-over study to evaluate objective physiological and subjective efficacy measures for arousal and desire of testosterone & sildenafil combination therapy in premenopausal women with acquired generalized female sexual interest & arousal disorder (FSIAD).

### Experimental Part

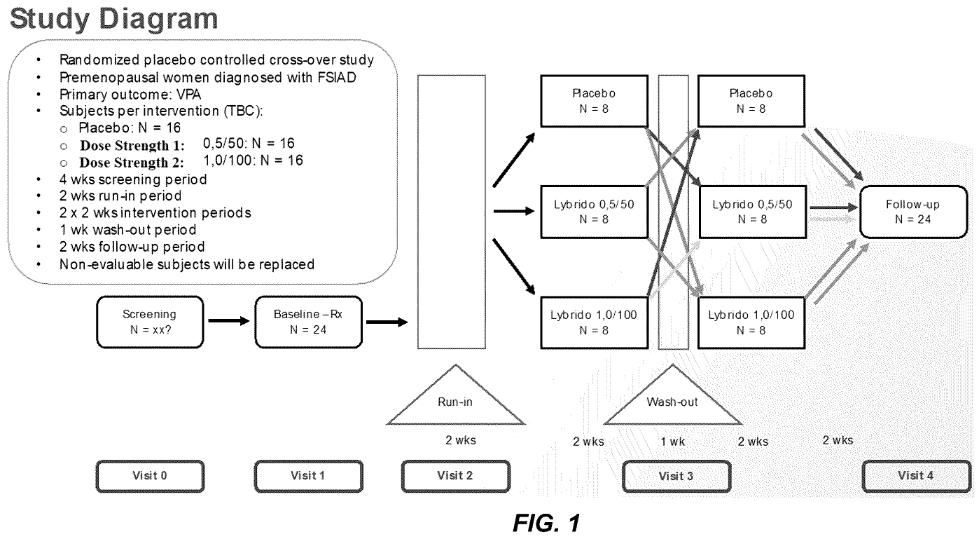

A study diagram is shown in FIG. 1. A schedule of event is provided in Table 8 below.

The study will involve a screening period of up to 4 weeks, a run-in period of 2 weeks, 2 treatment periods of 2 weeks, a wash-out period of 1 week, followed by a safety follow-up period of 2 weeks.

After the 4-week screening period eligible subjects will undergo baseline measurements, and will then be randomized to the following treatment sequences:
1. Placebo—T0,5/S50 (0.5 mg testosterone and 50 mg sildenafil) (n=4)
2. Placebo—T1,0/S100 (1.0 mg testosterone and 100 mg sildenafil) (n=4)
3. T0,5/S50—Placebo (n=4)
4. T0,5/S50—T1,0/S100 (n=4)
5. T1,0/S100—Placebo (n=4)
6. T1,0/S100—T0,5/S50 (n=4)

Subjects will be instructed regarding study procedures, including the VPP and VSS methods.

Subjects will be provided with a number of 6 dosings for each 2-week treatment period (2 tablets per dosing). Each subject is required to take 4 dosings over a 2-week period. Dosings are allowed to be taken as needed throughout the treatment period, with a maximum of 2 dosings per week and with at least 1 day between dosings.

Once at least 50% of subjects have been followed up for 2×2 weeks of treatment, an interim analysis will be performed overseen by an independent Data Monitoring Committee (DMC).

During the Screening visit (V0) subjects will be assessed according to the eligibility criteria. Subjects who meet all eligibility criteria will be randomized at the Baseline & Randomization visit (V1) to one of the 6 treatment sequences and baseline measurements will be performed.

After the Baseline and Randomization visit, subjects will first undergo a “Run-In period” of 2 weeks, as to familiarize with the VPP and VSS methods for at-home measurement similar as during the active treatment period, meaning performing 2 tests without taking study treatment, each week with at least 1 day between 2 tests, followed by the Pre-treatment visit (V2) as to verify correctness of applying the measurement (VPP and VSS) methods. During the Pre-treatment visit subjects will be instructed on the study drug administration procedure, and study medication for the 2 treatment periods of 2 weeks (6 dosings for each 2-week treatment period) will be dispensed (see under Study Drug, Dosage & Administration). During the Wash-out period an Interim Treatment Evaluation visit (by telephone if possible) (V3) will be planned, to verify protocol compliance and to answer subjects' questions. At the Safety Follow Up visit (V4), a final (safety) assessment will take place, and subjects will return all (unused) study medication and study materials to the investigational site.

Systematic (S)AE monitoring will be performed via a questionnaire during each visit. In addition, subjects will be requested to maintain an e-diary to report on several efficacy outcome measures as indicated in the SOE.

### Results

Results of Example 2 show that both dose strengths produced statistically significant increase in VPA over placebo, and the higher dose strength (1 mg testosterone and 100 mg sildenafil) achieved overall better results than the lower dose strength.

TABLE 8
Schedule of events

| | Period name | | | | | | | | | |
|---|---|---|---|---|---|---|---|---|---|---|
| | | | Run- | | | Wash- | | | | |
| | Screening | | in | | Treatment | out | Treatment | Follow- | | |
| | period | | period | | period 1 | period | period 2 | up period | | |
| | Visit name | | | | | | | | | |
| | | Baseline & | | End of | | | | | End of | |
| | Screening | Randomization | | Run- | | Mid Wash- | | | Study | Unscheduled |
| | visit | visit | | in visit | | out visit | | | visit | visit |
| | Visit number | | | | | | | | | |
| | 0 | 1 | | 2 | | 3 | | | 4 | |
| | Visit time | | | | | | | | | |
| | | | Day 0 | | Day 14 | Day 28 | Day 35 | | | |
| | Wk −8 | | to Day | | to Day | to Day | to Day | Day 49 | Day | |
| | to Day 0 | Day 0 | 14 | Day 14 | 28 | 35 | 49 | to 63 | 63 ^{b} | |
| | Days ± visit window since last visit | | | | | | | | | |
| Study Procedure | — | 56 ± 4 | | 14 ± 2 | | 18 ± 2 | | | 28 ± 4 | |
| ICF | X | | | | | | | | | |
| Demographics | X | | | | | | | | | |
| Medical history | X | | | | | | | | | |
| Prior/Concomitant | X | X | X | X | X | X | X | X | X | X |
| medication use | | | | | | | | | | |
| Adverse events | X | X | X | X | X | X | X | X | X | X |
| (assessed during site | | | | | | | | | | |
| visit; see also below | | | | | | | | | | |
| for e-diary) | | | | | | | | | | |
| Physical examination ^{c} | X | XX | | | | | | | X | |
| Weight | X | X | | | | | | | X | |
| Height | X | | | | | | | | | |
| Vital signs ^{d} | X | X | | | | X | | | X | X |
| Pregnancy (urine) (any | X | | | X | | X | | | X | X |
| positive urine | | | | | | | | | | |
| pregnancy test result is | | | | | | | | | | |
| to be confirmed | | | | | | | | | | |
| immediately by a | | | | | | | | | | |
| serum pregnancy test) | | | | | | | | | | |
| Hyperandrogenism | X | X | | | | X | | | X | |
| monitoring | | | | | | | | | | |
| Gynaecological | X | | | | | | | | | X |
| examination | | | | | | | | | | |
| Blood sample for | X | | | | | | | | [symbol] | X |
| clinical laboratory ^{d} | | | | | | | | | | |
| FSH (if | X | | | | | | | | | |
| needed, see | | | | | | | | | | |
| excl. 17) | | | | | | | | | | |
| Oestradiol (if | X | | | | | | | | | |
| needed, see | | | | | | | | | | |
| excl. 17) | | | | | | | | | | |
| Testosterone | X | | | | | | | | X | |
| (total and | | | | | | | | | | |
| free) | | | | | | | | | | |
| SHBG | X | | | | | | | | [symbol] | |
| Potential | X | | | | | | | | [symbol] | |
| future | | | | | | | | | | |
| biomarker | | | | | | | | | | |
| analysis | | | | | | | | | | |
| Breast examination ^{e} | X | | | | | | | | | X |
| Mammography ^{e} | X | | | | | | | | | |
| Pelvic | X | | | | | | | | | |
| examination/vaginal | | | | | | | | | | |
| cultures | | | | | | | | | | |
| Pap smear | X | | | | | | | | | |
| Randomization | | X | | | | | | | | |
| Instruct/check on | | X | | X | | X | | | | X |
| dosing procedure ^{f} | | | | | | | | | | |
| Instruct on/get used to | X | X | | X | | X | | | | X |
| correct completion e- | | | | | | | | | | |
| diary | | | | | | | | | | |
| Instruction on the use | | X | | | | | | | | |
| of the VPA | | | | | | | | | | |
| measurement | | | | | | | | | | |
| Dispense and/or | | | | X | | X | | | X | |
| collect study drug | | | | | | | | | | |
| Take study medication | | | | | X ^{g} | | X ^{g} | | | |
| at home as needed and | | | | | | | | | | |
| record in e-diary ^{g} | | | | | | | | | | |
| Completion of e- | | | | | | | | | | |
| diary: | | | | | | | | | | |
| Systematic | X | X | X | X | X | X | X | X | X | X |
| (S)AE | | | | | | | | | | |
| monitoring ^{h} | | | | | | | | | | |
| FSFI | X ⁱ | X | X | | X | X | X | X | | |
| FSDS-DOA | X ⁱ | X | X | | X | X | X | X | | |
| Sexual | | | X ^{j} | | | | | | | |
| episode | | | | | | | | | | |
| question ^{j} | | | | | | | | | | |
| Dosing | | | X ^{j} | | | | | | | |
| question ^{j} | | | | | | | | | | |
| EDQ ‘event- | | | X ^{k} | | | | | | | |
| based’ ^{k} | | | | | | | | | | |
| Alcohol use | | | X | | | | | | | |
| Patient | | | X | | X | X | X | X | | |
| Global | | | | | | | | | | |
| Impression of | | | | | | | | | | |
| Improvement | | | | | | | | | | |
| Check/Confirm | X | X | | | | | | | | |
| Eligibility Criteria | | | | | | | | | | |

Abbreviations: AE = adverse event; D = Day; EDQ = Elements of Desire Questionnaire; EOS = End of Study; EOT = End of Treatment; excl. = exclusion; FSDS- DOA = Female Sexual Distress Scale - Desire, Orgasm, Arousal; hr = hour; PGI-C = Patient Global Impressions of Change; SHBG = Sex Hormone Binding Globulin; SSEQ = Sexual Satisfaction of an Event Questionnaire; Unsch = unscheduled.; Wk = Week (7 days); X = to be assessed.

a. The screening period of up to 4 weeks will be followed by a 4-week baseline period without study drug treatment, to measure baseline values for a selection of (secondary) efficacy endpoints.
b. The EOS visit is a phone call visit.
c. A full examination will be performed as indicated; a symptom-directed examination will be performed at all other visits.
d. Assessed via central lab with the exception of assessments which are only necessary for the purpose of eligibility assessment (ie, HIV, serum hCG test, FSH, drug/alcohol screen) which can be assessed through a local lab.
e. The breast examination is a careful manual examination of the breasts and will be performed at screening, end of treatment, and in case of any concern of the subject. Mammography will only be performed during screening for women >40 years of age and in case of a family history of breast cancer cases and only in the absence of a documented normal mammography result performed within 12 months of screening. During the treatment period a mammography will only be performed upon findings from the manual breast examination.
f. Medication to be taken as needed, 3-6 hour prior to anticipated sexual activity. The minimum intake of study drug is 4 times per 4 weeks, the maximum intake is 2 times/week with at least 1 day apart. The exact timing of each medication intake is to be recorded in the diary.
g. Dosing should take place after some food intake. Each tablet should be held at least 90 seconds under the tongue. More detailed instructions will be provided per the instructions for use for the patient.
h. Systematic (S)AE monitoring will be performed through a questionnaire at each visit, and includes monitoring for hyperandrogenism, visual disturbances, etc. At each visit subjects will be instructed to contact the investigator if their menses is delayed more than 14 days.
i. During the screening visit the FSFI and the FSDS-DOA will not be assessed through the e-diary and will only be used for the purpose of checking the results versus the cut-off values listed in the eligibility criteria.
j. At a daily basis the question is asked whether a sexual episode (ie, any act involving sexual contact with genitalia and/or oral mucosa and includes caressing, foreplay, intercourse, oral sex, anal sex, and masturbation [by self or partner] or dosing occurred, along with a question on alcohol use. If yes, the timing of sexual episode or dosing needs to be recorded along with an evaluation of satisfaction via the SSEQ, and completion of the EDQ ‘event-based’ (24 hr recall).
k. The EDQ ‘event-based’ is to be completed 24 hour after an ‘event’ occurs, whereby an ‘event’ is defined as a sexual episode (ie, any act involving sexual contact with genitalia and/or oral mucosa and includes caressing, foreplay, intercourse, oral sex, anal sex, and masturbation [by self or partner] or the actual dosing of the study drug.

### Example 3

The experiment of Example 3 is as described in a previous publication (Tuiten et al, The Journal of Sexual Medicine, 2018:15, 201-216), the content of which is incorporated herein.

### Study Design and Oversight

Double-blinded, randomized, placebo-controlled, dose-finding phase 2 studies (identical in study design) were conducted at 16 sites in the United States. Data were collected, managed, and analyzed under the supervision of the sponsor, which was monitored by Rasmussen Biotech and Pharma Consulting LLC (Princeton, NJ, USA). The participants, investigators, and site personnel were blinded to study treatment until database lock, as were the sponsor and those members of the vendors' staff who were involved (eg, site monitors). The first study started on Sep. 27, 2011 (first screening visit) and the final study ended on Sep. 26, 2014 (database lock). The PSR-Group (Hoofddorp, The Netherlands) was responsible for data management. The trials are registered under identifiers NCT01432665, NCT01743235, and NCT02101203 (ClinicalTrials.gov).

### Study Population

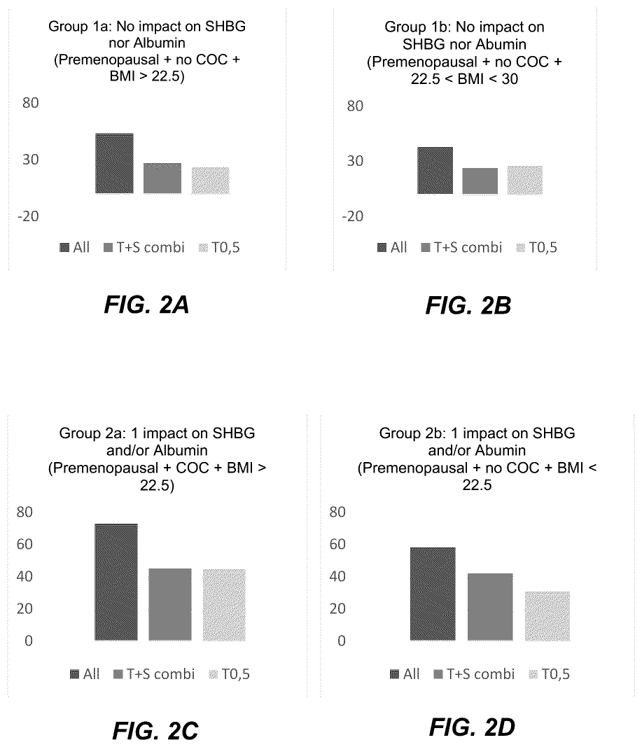

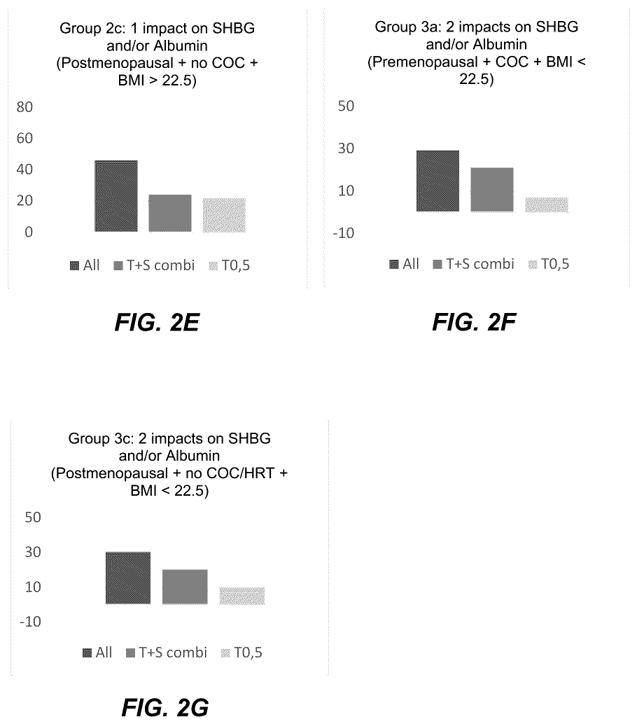

The requisite sample size was determined by a power analysis, which took account of the effect sizes found in previous studies. The female participants included in this study were 21 to 70 years old and had been diagnosed with HSDD (based on DSM-IV-TR criteria),32 with or without FSAD, but were otherwise healthy. Our inclusion criteria also were consistent with the current DSM-5 diagnosis of FSIAD. All diagnoses were performed by a trained professional. A demarcation formula was used to assign participants to 1 of the 2 FSIAD subgroups (see text and Tables 1-3 and FIGS. 1 and 2 of Tuiten). Participants who had been diagnosed with any psychiatric disorder other than FSIAD were excluded. Those scheduled for any other treatment for female sexual dysfunction during the study period also were excluded. Potential participants were excluded if they were using oral contraceptives containing anti-androgens, estrogen more than 50 mg, potent cytochrome P450 3A4 inhibitors or inducers, nitrates, monoamine oxidase inhibitors, antidepressants, and/or T compounds. The cardiovascular exclusion criteria included a history of myocardial infarction, stroke, or life-threatening arrhythmia in the previous 6 months. Other criteria were uncontrolled hypertension, hypotension, atrial fibrillation or flutter, or any other significant abnormality observed on an electrocardiogram. The gynecologic exclusion criteria included pelvic inflammatory disease, vaginal infection, previous prolapse and incontinence surgery affecting the vaginal wall, abnormal uterine bleeding patterns, pregnancy, and breastfeeding in the past 6 months. Perimenopausal women were excluded, which was defined as cycle shortening or irregular menstrual bleeding in the past 12 consecutive months and/or occurrence of vasomotor symptoms (eg, hot flashes, nocturnal sweats) in combination with increased follicle-stimulating hormone levels (>40 IU/L) for women at least 40 years old; for women with a history of hysterectomy, perimenopausal status was determined by assessing follicle-stimulating hormone levels (>40 IU/L) and/or vasomotor symptoms. Women with clinically relevant endocrine disease, neurologic disease, severe or acute liver disease, or a history of severe hepatic impairment were excluded. Participants were excluded if they had free and/or total T levels that were beyond the upper limit of the central laboratory's reference range. All participants provided written informed consent.

497 women were randomized to 1 of the 12 treatment regimens: placebo, the monotherapies (T 0.5 mg, S 50 mg, and B 10 mg), and 4 types of combination tablets (containing different dose combinations). In participants with low levels of sensitivity, the doses of T+S (milligrams) were T0.25+525, T0.25+S50,T0.5+S25, and T0.5+S50.

### Study Procedures

After a baseline period of 4 weeks, participants underwent an 8-week, single-blinded, placebo run-in (PRI) period, which was intended to stabilize placebo responses before active treatment. The PRI period was followed by an 8-week, double-blinded ATP. Those participants who according to the demarcation formula had low sensitivity (and therefore allocated to T+S treatments) were randomly assigned to 1 of the following treatment arms: (i) placebo, (ii) S 50 mg, (iii) T 0.50 mg, (iv) T0.25 mg+S25 mg, (v) T0.25 mg+S50 mg, (vi) T0.50 mg+S25 mg, or (vii) T0.50 mg+S50 mg. All dose combinations, including the placebo, were contained in combination tablets that were identical in size, taste, texture, and odor. The participants were instructed to take the study medication approximately 3 to 6 hours before they anticipated engaging in sexual activity (ie, on-demand use). Each participant was issued with 28 U of medication during each 8-week treatment period. They were instructed to allow a minimum period of at least 48 hours between individual doses.

The participants were interviewed every 4 weeks, at which time they also completed questionnaires designed to assess sexual function and mood. In addition, their vital signs were measured, any signs of hyperandrogenism were evaluated, a pregnancy test was administered, and any adverse events were monitored. A psychological interview was conducted at weeks 8 and 16 to evaluate each 8-week treatment period, at which time blood samples were collected to assess each participant's chemistry, hematology, lipids, and hormones.

### Medication, Dosing, and Instructions

Sublingual Testosterone+Sildenafil (T+S). This drug is a dual-route, dual-release, fixed-dose combination of T and S citrate. The drug product is a 9-mm, round, biconvex, white, menthol-flavored tablet for sublingual administration. The outer coating (a polymeric film) contains T (0.5 mg) that is released immediately at sublingual administration. The inner core of the tablet, which contains S (50 mg), has a polymeric coating designed to delay the release of that drug for approximately 2.5 hours. When that period elapses, S is released immediately (ie, there is no sustained release).

The placebo tablets were identical in appearance and flavor to the fixed-dose combination T+S tablets containing the active pharmaceutical ingredients. All study medication was manufactured and packaged at Piramal Healthcare UK (Morpeth, UK).

Participants were instructed to keep the tablet under their tongues for 90 seconds and then to swallow it whole, without chewing it or otherwise disrupting the dosage form. They were permitted to take the tablet with a little water. The participants were instructed to engage in sexual activity 3 to 6 hours after ingestion. The dosing instructions were the same for all regimes.

28 doses were provided in the 8-week treatment period. The participants were asked to endeavor to take a minimum of 1 dose per week. They were informed that they could take the remaining doses as required (ie, on demand) throughout the 8-week treatment period. The minimum period between individual doses was 2 days (ie, on alternate days).

### Randomization and Masking

Participants who met the inclusion and exclusion criteria were randomized and allocated to a treatment sequence. The randomization list was designed by an independent statistician at Pharma Consulting Group (Uppsala, Sweden). Randomization was performed using an interactive web response system (Viedoc; Pharma Consulting Group), with a unique numeric medication code from the randomization list being assigned to each randomized subject. The corresponding medication kit (bearing the same unique number) was sent to the site by the warehouse where the study medication was stored (Sentry BioPharma Services Inc, Indianapolis, IN, USA). The factors body mass index and menopausal status (pre- or postmenopausal) were balanced across treatment arms. A unique subject identification code was used to ensure that the data were anonymous. Access to the key to this identification code was restricted. An 8-week, single-blinded PRI period was used to dampen the usual placebo effect during active treatments. Investigators were aware that all participants received placebo during the PRI period, but the participants were not. The investigators and site personnel involved were given appropriate instruction and training to minimize potential effects of this knowledge. The blinding could be broken only under exceptional circumstances, for example, if the investigator believed that it was vital for the medical management of the participant in question. In such cases, investigators were required to contact the sponsor's medical monitor who had access to the sealed code envelopes.

### Outcomes and Assessments

Primary and secondary outcome variables were measured using the validated Sexual Event Diary (SED). The SED is a secure web-based diary that participants were required to complete within a period of 24 hours after each sexual event. This information was used to assess each participant's satisfaction and sexual functioning during that event. Participants were asked to indicate whether the event was satisfactory and whether they experienced an orgasm (dichotomous items). They also were asked to indicate the associated levels of sexual desire, pleasure, bodily arousal, and subjective arousal (using 5-point Likert scale items). In addition, they were asked for details of the type and duration of the sexual event in question and whether they used the study medication before that event.

The primary end point was the change In the number of SSEs (as recorded by the SED) in the interval from the 4-week baseline to the 4-week mean of the 8-week ATP. This end point is one of the FDA's accepted primary end points for clinical trials in HSDD and FSIAD. The following formula (Formula 1) was used to calculate change in the number of SSEs from baseline (DSSE) for each participant:

$$Δ SSE = \frac{( \sum {SSE}_{ATP 1 - 4} + \sum {SSE}_{ATP 5 - 8} )}{2} - ( \sum {SSE}_{BLE 1 - 4} )$$

where ATP1-4 represents ATP weeks 1 through 4, ATP5-8 represents ATP weeks 5 through 8, and BLE1-4 represents baseline establishment at weeks 1 through 4. The key secondary end point was the change (during the same interval) in the cumulative level of subjective sexual satisfaction (4-item mean of desire, pleasure, bodily arousal, and subjective arousal) during the SSEs produced by the treatments. Another secondary end point was the change in the number of orgasms (as recorded by the SED) from the 4-week baseline to the 4-week mean of the 8-week ATP. The PRI period was not used in the analyses because it was intended only to stabilize placebo responses.

Additional secondary outcome variables were assessed using the Weekly Diary (WD). Each week, participants filled out the WD at home using the same secure web-based platform as the SED. The WD assessed how often participants experienced sexual desire and arousal during the preceding week, ranging from “not at all” to “more than a couple of times a day” on a 6-point scale. Also, subjects were asked whether the perceived change was caused by the study medication.

Efficacy of the treatment was evaluated by assessing responses to questionnaires measuring sexual function, satisfaction and distress associated with sexual function. In particular, the questionnaire contains questions on ‘Pleasurable (Q5)’, ‘Mood for sex/Sexual desire (Q7)’, ‘Bodily arousal (Q8)’, ‘Sexually aroused (Q9)’, ‘Feel good about body (Q10)’, ‘Feelings of disgust (Q13)’, ‘Let go (Q6)’, ‘Afraid (Q11)’, ‘Distracting thoughts (Q13), ‘Partner (Q14, Q15)’, and ‘Orgasm (Q16)’.

### Statistical Analyses

To assess the primary end point, interaction effects were calculated among the highest-dose combinations, the placebo condition, and the monotherapies (S and T alone). This allowed the comparison of the change in the number of SSEs from the 4-week baseline establishment period to the 4-week mean of the 8-week ATP. The primary end point and the method of analysis were agreed upon with the regulatory agencies (FDA and European Medicines Agency [EMA]). A similar approach was used to assess the key secondary end point and the other secondary end points. The interaction effects were derived by using regression analyses on each imputed dataset (see supporting information for a description of the imputation methods). The difference between baseline and active treatment was used as the dependent variable and the treatment group indicator was used as the independent variable. The regression coefficient of the treatment group indicator represents the difference in change from baseline between the 2 treatment groups. Regression coefficient estimates and standard errors were combined according to Rubin's Rules.

The p value was set at 0.05 (2-sided). Statistical analyses were performed in line with the statistical analysis plan as described in the clinical study protocols (see supporting information; note that the SED was formerly named SSEQ) using IBM SPSS Statistics 21.0 for Windows (IBM Corp, Armonk, NY, USA) and R 3.2 (The R Project for Statistical Computing, The R Foundation, Vienna, Austria).

The safety analysis population consisted of all those participants who received at least 1 dose of study medication. Descriptive statistics were calculated for all safety data. All adverse events were listed, as were the number of participants in each treatment group involved and details of the incidence of these events. For the incidence assessment, cases in which a single participant experienced adverse events on more than 1 occasion were counted as single incidents.

### Results and Observations:

Participants are categorized into four different groups based on the criteria below:
1) Group 1: No impact on SHBG nor Albumin
   1a: Premenopausal+no COC+BMI >22.5
   1b: Premenopausal+no COC+22.5<BMI <30
2) Group 2: 1 impact on SHBG and/or Albumin:
   2a: Premenopausal+COC+BMI >22.5
   2b: Premenopausal+no COC+BMI <22,5
   2c: Postmenopausal+no COC/HRT+BMI >22,5
3) Group 3: 2 impacts on SHBG and/or Albumin:
   3a: Premenopausal+COC+BMI <22.5
   3b: Postmenopausal+COC/HRT+BMI >22.5
   3c: Postmenopausal+no COC/HRT+BMI <22.5
4) Group 4: 3 impacts on SHBG and/or Albumin:
   4a: Postmenopausal+COC/HRT+BMI <22.5.

The numbers of participants in each (sub)group and the percentages of (those) participants showing improvement in any arm (including the placebo arm), in the combination therapy arm, and in the monotherapy (T only) arm, are shown in Table 9.

TABLE 9
stratification of participants

| Group | Total Participants | Any | T + S Combi | T only |
|---|---|---|---|---|
| 1a | 78 | 53% | 27% | 24% |
| 1b | 46 | 43% | 24% | 26% |
| 2a | 11 | 73% | 45% | 45% |
| 2b | 26 | 58% | 42% | 31% |
| 2c | 54 | 46% | 24% | 22% |
| 3a | 14 | 29% | 21% | 7% |
| 3b | 0 | N/A | N/A | N/A |
| 3c | 10 | 30% | 20% | 10% |
| 4a | 0 | N/A | N/A | N/A |

FIGS. 2A to 2G are diagrams are bar charts based on the data included in Table 9.

### Observations:

1) The number of participants falling in a group decreases with increasing number of impacts of the group;
2) In premenopausal women the type of impact (COC use versus BMI <22,5) makes no significant difference on the efficacy and effects of the treatment.
3) Impact of (only) menopause seems to have the same magnitude as (as influential as) 2 impacts (COC use plus BMI <22,5) in premenopausal women.
4) High BMI (>30) has no impact on percentage of responders, and
5) Increasing the testosterone dose seems to broaden the window of opportunity to increase the percentage of responders, in premenopausal women and even more in postmenopausal women.

Exemplary participants and results thereof from each (sub)group are further analyzed. In results shown below, level of SHBG is expressed in nmol/L, level of albumin is expressed in g/dL, total and free testosterone levels are expressed in ng/dL, follicle-stimulating hormone (FSH) level is expressed in IU/L. Scores for Q7 (Sexual Desire), Q8 (Bodily Arousal), Q9 (Sexually Aroused), the Desire Domain (composite of Q7 and Q9), the Satisfaction Domain (the mean of Q5-Q9 plus Q11-Q13) and SSE (calculated according to Formula I) are analyzed.

Response in the placebo arm is used as a reference point:

| Treatment: | placebo |
|---|---|
| Menopause: | − |
| COC/HRT: | − |
| BMI: | >22.5 |
| SHBG: | 75.8 |
| Albumin: | 4.5 |
| Total Testo; | 29.0 |
| Free Testo: | 0.21 |
| FSH: | 8.6 |
| Q7: | +0.6 |
| Q8: | +0.7 |
| Q9: | +0.8 |
| Desire Domain: | +0.7 |
| Satisfaction: | +3.0 |
| SSE: | +0.2 |

Exemplary results of participants in Group 1 treated with 0.5 mg testosterone in combination with 50 mg sildenafil

| | | | | | |
|---|---|---|---|---|---|
| Patient ID: | 001-029 | Patient ID: | 002-015 | Patient ID: | 015-006 |
| Treatment: | 0.5/50 | Treatment: | 0.5/50 | Treatment: | 0.5/50 |
| Menopause: | − | Menopause: | − | Menopause: | − |
| COC/HRT: | − | COC/HRT: | − | COC/HRT: | − |
| BMI: | 34.61 | BMI: | 26.43 | BMI: | 27.72 |
| SHBG: | 28.0 | SHBG: | 61.7 | SHBG: | 174.7 |
| Albumin: | 4.3 | Albumin: | 3.9 | Albumin: | 4.0 |
| Total Testo: | 54.0 | Total Testo: | 16.0 | Total Testo: | 34.0 |
| Free Testo: | 1.07 | Free Testo: | 0.19 | Free Testo: | 0.17 |
| FSH: | 8.5 | FSH: | 10.0 | FSH: | 1.6 |
| Q7: | +2.35 | Q7: | +3.25 | Q7: | +2.45 |
| Q8: | +2.08 | Q8: | +3.25 | Q8: | +3.92 |
| Q9: | +2.60 | Q9: | +3.00 | Q9: | +3.76 |
| Desire | +2.48 | Desire | +3.13 | Desire | +3.11 |
| Domain: | | Domain: | | Domain: | |
| Satisfaction: | +13.98 | Satisfaction: | +15.05 | Satisfaction: | +18.88 |
| SSE: | +2.5 | SSE: | +2.0 | SSE: | +6.5 |

Exemplary results of participants in Group 2a treated with 0.5 mg testosterone, optionally with 25 mg or 50 mg sildenafil

| | | | | | |
|---|---|---|---|---|---|
| Patient ID: | 003-021 | Patient ID: | 007-017 | Patient ID: | 008-002 |
| Treatment: | 0.5/0 | Treatment: | 0.5/25 | Treatment: | 0.5/25 |
| Menopause: | − | Menopause: | − | Menopause: | − |
| COC/HRT: | + | COC/HRT: | + | COC/HRT: | + |
| BMI: | 22.90 | BMI: | 26.94 | BMI: | 30.04 |
| SHBG: | 183.7 | SHBG: | 151.8 | SHBG: | 153.0 |
| Albumin: | 4.8 | Albumin: | 4.8 | Albumin: | 4.0 |
| Total Testo: | <10.0 | Total Testo: | 30.0 | Total Testo: | 26.0 |
| Free Testo; | <0.05 | Free Testo: | 0.17 | Free Testo: | 0.15 |
| FSH: | 5.9 | FSH: | 11.5 | FSH: | 0.2 |
| Q7: | +0.58 | Q7: | +2.13 | Q7: | +1.33 |
| Q8: | +0.67 | Q8: | +2.25 | Q8: | +0.33 |
| Q9: | +0.58 | Q9: | +2.5 | Q9: | +0 |
| Desire | +0.58 | Desire | +2.19 | Desire | +0.67 |
| Domain: | | Domain: | | Domain: | |
| Satisfaction: | +3.75 | Satisfaction; | +9.13 | Satisfaction; | +4.33 |
| SSE: | +2.5 | SSE: | +2.0 | SSE: | +0.5 |

| | | | |
|---|---|---|---|
| Patient ID: | 016-020 | Patient ID: | 017-012 |
| Treatment: | 0.5/50 | Treatment: | 0.5/25 |
| Menopause: | − | Menopause: | − |
| COC/HRT: | + | COC/HRT: | + |
| BMI: | 24.01 | BMI: | 28.29 |
| SHBG: | 183.2 | SHBG: | 203.8 |
| Albumin: | 4.5 | Albumin: | 4.2 |
| Total Testo: | 49.0 | Total Testo: | 29.0 |
| Free Testo: | 0.24 | Free Testo: | 0.13 |
| FSH: | 5.0 | FSH: | 10.3 |
| Q7: | +0.29 | Q7: | +2.25 |
| Q8: | +0.96 | Q8: | +1.0 |
| Q9: | +0.75 | Q9: | +1.0 |
| Desire | +0.52 | Desire | +1.63 |
| Domain: | | Domain: | |
| Satisfaction: | +3.50 | Satisfaction: | +6.50 |
| SSE: | +0.5 | SSE: | −3.0 |

Exemplary results of participants in Group 2b treated with 0.5 mg testosterone, optionally with 25 mg or 50 mg sildenafil

| | | | | | |
|---|---|---|---|---|---|
| Patient ID: | 002-021 | Patient ID: | 008-014 | Patient ID: | 008-051 |
| Treatment: | 0.5/25 | Treatment: | 0.5/25 | Treatment: | 0.5/50 |
| Menopause: | − | Menopause: | − | Menopause: | − |
| COC/HRT: | − | COC/HRT: | − | COC/HRT: | − |
| BMI: | 21.48 | BMI: | 18.91 | BMI: | 18.45 |
| SHBG: | 88.4 | SHBG: | 127.5 | SHBG: | 63.5 |
| Albumin: | 5.0 | Albumin: | 4.7 | Albumin: | 4.6 |
| Total Testo: | 13.0 | Total Testo: | 30.0 | Total Testo: | 52.0 |
| Free Testo: | 0.11 | Free Testo: | 0.20 | Free Testo: | 0.6 |
| FSH: | 41.5 | FSH: | 2.2 | FSH: | 3.4 |
| Q7: | −2.5 | Q7: | −0.25 | Q7: | +0.63 |
| Q8: | −2.5 | Q8: | +0.33 | Q8: | +0.75 |
| Q9: | −1.0 | Q9: | +0 | Q9: | +0.5 |
| Desire | −1.75 | Desire | −0.13 | Desire | +0.56 |
| Domain: | | Domain: | | Domain: | |
| Satisfaction: | −11.5 | Satisfaction: | −2.0 | Satisfaction: | +0.75 |
| SSE: | −0.5 | SSE: | −1.5 | SSE: | +0 |

Exemplary results of participants in Group 2c treated with 0.5 mg testosterone, optionally with 25 mg or 50 mg sildenafil

| | | | | | |
|---|---|---|---|---|---|
| Patient ID: | 006-006 | Patient ID: | 013-024 | Patient ID: | 014-032 |
| Treatment: | 0.5/0 | Treatment: | 0.5/25 | Treatment: | 0.5/50 |
| Menopause: | + | Menopause: | + | Menopause: | + |
| COC/HRT: | − | COC/HRT: | − | COC/HRT: | − |
| BMI: | 37.07 | BMI: | 30.87 | BMI: | 31.30 |
| SHBG: | 27.3 | SHBG: | 36.6 | SHBG: | 15.9 |
| Albumin: | 4.6 | Albumin: | 4.6 | Albumin: | 4.7 |
| Total Testo: | 55.0 | Total Testo: | 25.0 | Total Testo: | <10.0 |
| Free Testo: | 1.10 | Free Testo: | 0.4 | Free Testo: | <0.24 |
| FSH: | 33.7 | FSH: | 20.3 | FSH: | 27.9 |
| Q7: | +0.31 | Q7: | −0.07 | Q7: | −0.90 |
| Q8: | +0.19 | Q8: | +0.47 | Q8: | −0.22 |
| Q9: | −0.13 | Q9: | +0.19 | Q9: | −0.07 |
| Desire | +0.09 | Desire | +0.06 | Desire | −0.49 |
| Domain: | | Domain: | | Domain: | |
| Satisfaction: | +0.81 | Satisfaction: | +0.36 | Satisfaction: | +0 |
| SSE: | +1.5 | SSE: | +1.0 | SSE: | +1.0 |

| | | | |
|---|---|---|---|
| Patient ID: | 014-041 | Patient ID: | 014-095 |
| Treatment: | 0.5/25 | Treatment: | 0.5/25 |
| Menopause: | − | Menopause: | − |
| COC/HRT: | − | COC/HRT: | − |
| BMI: | 25.18 | BMI: | 30.82 |
| SHBG: | 27.6 | SHBG: | 22.5 |
| Albumin: | 4.4 | Albumin: | 4.7 |
| Total Testo: | <10.0 | Total Testo: | 17.0 |
| Free Testo: | <0.20 | Free Testo: | 0.36 |
| FSH: | 55.5 | FSH: | 83.4 |
| Q7: | −0.54 | Q7: | +0 |
| Q8: | +0.17 | Q8: | +0 |
| Q9: | −0.08 | Q9: | +0.38 |
| Desire | −0.31 | Desire | +0.19 |
| Domain: | | Domain: | |
| Satisfaction: | +1.58 | Satisfaction: | +0.5 |
| SSE: | +1.5 | SSE: | +1.0 |

Exemplary results of participants in Group 3a treated with 0.5 mg testosterone, optionally with 25 mg or 50 mg sildenafil

| | | | | |
|---|---|---|---|---|
| Patient ID: | 008-005 | Patient ID: | 008-050 | Patient ID: |
| Treatment: | 0.5/25 | Treatment: | 0.5/50 | Treatment: |
| Menopause: | − | Menopause: | − | Menopause: |
| COC/HRT: | + | COC/HRT: | + | COC/HRT: |
| BMI: | 21.92 | BMI: | 19.75 | BMI: |
| SHBG: | 113.2 | SHBG: | 157.5 | SHBG: |
| Albumin: | 4.2 | Albumin: | 4.6 | Albumin: |
| Total Testo: | <10.0 | Total Testo: | 42.0 | Total Testo: |
| Free Testo: | <0.07 | Free Testo: | 0.23 | Free Testo: |
| FSH: | 0.5 | FSH: | 4.3 | FSH: |
| Q7: | +0.67 | Q7: | +0.67 | Q7: |
| Q8: | +1.63 | Q8: | +1.67 | Q8: |
| Q9: | +1.69 | Q9: | +1.50 | Q9: |
| Desire Domain: | +1.18 | Desire Domain: | +1.08 | Desire Domain: |
| Satisfaction: | +7.71 | Satisfaction: | +6.33 | Satisfaction: |
| SSE: | +6.0 | SSE: | +0 | SSE: |

Exemplary results of participants in Group 3c treated with 0.5 mg testosterone, optionally with 25 mg or 50 mg sildenafil

| | | | | | |
|---|---|---|---|---|---|
| Patient ID: | 001-023 | Patient ID: | 009-023 | Patient ID: | 012-009 |
| Treatment: | 0.5/25 | Treatment: | 0.5/50 | Treatment: | 0.5/0 |
| Menopause: | + | Menopause: | + | Menopause: | + |
| COC/HRT: | − | COC/HRT: | − | COC/HRT: | − |
| BMI: | 22.35 | BMI: | 17.64 | BMI: | 20.38 |
| SHBG: | 95.4 | SHBG: | 100.8 | SHBG: | 99.2 |
| Albumin: | 4.9 | Albumin: | 4.5 | Albumin: | 4.5 |
| Total Testo: | 68.0 | Total Testo: | 21.0 | Total Testo: | 32.0 |
| Free Testo: | 0.57 | Free Testo: | 0.17 | Free Testo: | 0.26 |
| FSH: | 97.3 | FSH: | 53.2 | FSH: | 70.2 |
| Q7: | +1.0 | Q7: | +0.87 | Q7: | +0.83 |
| Q8: | +0.13 | Q8: | +0.50 | Q8: | +0.70 |
| Q9 | +0.45 | Q9: | +0.37 | Q9: | +0.52 |
| Desire | +0.73 | Desire | +0.62 | Desire | +0.68 |
| Domain: | | Domain: | | Domain: | |
| Satisfaction: | +1.48 | Satisfaction: | +2.73 | Satisfaction: | +1.35 |
| SSE: | +0 | SSE: | +1.0 | SSE: | −2.0 |

Exemplary results of pre-menopausal participants having a plasma SHBG level of high than 100 nmol/L, treated with 0.5 mg testosterone alone.

| | | | | | |
|---|---|---|---|---|---|
| Patient ID: | 009-042 | Patient ID: | 014-009 | Patient ID: | 014-088 |
| Treatment: | 0.5/0 | Treatment: | 0.5/0 | Treatment: | 0.5/0 |
| Menopause: | − | Menopause: | − | Menopause: | − |
| COC/HRT: | − | COC/HRT: | − | COC/HRT: | − |
| BMI: | 27.45 | BMI: | 24.79 | BMI: | 24.16 |
| SHBG: | 155.0 | SHBG: | 104.9 | SHBG: | 167.5 |
| Albumin: | 4.8 | Albumin: | 4.4 | Albumin: | 4.3 |
| Total Testo: | 59.0 | Total Testo: | 15.0 | Total Testo: | 37.0 |
| Free Testo: | 0.33 | Free Testo: | 0.12 | Free Testo: | 0.20 |
| FSH: | 3.1 | FSH: | 14.0 | FSH: | 0.4 |
| Q7: | +1.1 | Q7: | −0.5 | Q7: | +0.56 |
| Q8: | +0.75 | Q8: | −0.75 | Q8: | +0.17 |
| Q9: | +1.33 | Q9: | −0.5 | Q9: | +0.25 |
| Desire | +1.21 | Desire | −0.5 | Desire | +0.41 |
| Domain: | | Domain: | | Domain: | |
| Satisfaction: | +5.08 | Satisfaction: | −5.75 | Satisfaction: | +2.21 |
| SSE: | +0.5 | SSE: | −1.0 | SSE: | =2.0 |

### Discussion of the Results:

With increasing the number of “impact actors,” the improvement by T0,5+/−S on sexual function outcomes appear to have decreased. Menopausal status seems to be the strongest (negative) impact factor on the efficacy of the treatment. In pre-menopausal women, it was noted that COC medication has given rise to strong increase in SHBG levels, and low BMI also has, in some cases, impacted the SHBG level. COC medication in combination with low BMI gave strong increase in SHBG level in pre-menopausal women. Low BMI had shown some impact on SHBG level in post-menopausal women. The results show that high SHBG levels, as well as being post-menopausal, has a strong (negative) impact on sexual function outcomes of the treatments, indicating that a higher testosterone dosage may be required.

Embodiments of the disclosure are described in the following paragraphs.

1. A dual release drug delivery system for use in enhancing female sexual desire in the treatment of Female Sexual Interest and Arousal Disorder (FSIAD), the composition comprising:
a core comprising cellulose, a filler selected from an organic and/or an inorganic salt, and a first active ingredient for delayed immediate release;
a first coating surrounding the core, the first coating comprising a hydrophobic polymer and a hydrophilic substance; and
a second coating surrounding the first coating, the second coating comprising a second active ingredient for immediate release;
wherein:
the first active ingredient comprises a PDE5 inhibitor;
the second active ingredient is a testosterone, or a functional analogue or derivative of testosterone,
the delayed release of the first active ingredient is an immediate release occurring between 2 to 6 hours, preferably between 2.5 to 3.5 hours, after the release of the second active ingredient; and wherein:
the second active ingredient is present in an amount equivalent to from 0.3 to 1.5 mg of testosterone.

2. The dual release drug delivery system for use according to embodiment 1, wherein the PDE5 inhibitor is selected from the group consisting of sildenafil, tadalafil, vardenafil, and combinations thereof, for example, the first active ingredient comprises sildenafil.

3. The dual release drug delivery system for use according to embodiment 1 or 2, wherein the first active ingredient is sildenafil, present in an amount of from 10 to 120 mg, preferably from 20 to 100 mg, for example 25 mg or 50 mg or 100 mg.

4. The dual release drug delivery system for use according to any one of the preceding embodiments, wherein the first active ingredient is to be administered orally.

5. The dual release drug delivery system for use according to any one of the preceding embodiments, wherein the second active ingredient is to be administered sublingually.

6. The dual release drug delivery system for use according to any one of the preceding embodiments, wherein the filler is an inorganic salt, for example anhydrous dibasic calcium phosphate.

7. The dual release drug delivery system for use according to any one of the preceding embodiments, wherein the cellulose is microcrystalline cellulose.

8. The dual release drug delivery system for use according to any one of the preceding embodiments, wherein the core further comprises a water-insoluble gel-forming disintegrant comprising cross-linked sodium carboxy methylcellulose, sodium starch glycolate and/or cross-linked polyvinylpyrrolidone.

9. The dual release drug delivery system for use according to any one of the preceding embodiments, wherein the hydrophobic polymer is a hydrophobic polymeric ethylcellulose.

10. The dual release drug delivery system for use according to any one of the preceding embodiments, wherein the hydrophilic substance is a water insoluble hydrophilic substance and the first coating contains pores prior to exposure to an aqueous liquid; or wherein the hydrophilic substance is a water soluble hydrophilic substance and the water soluble hydrophilic substance forms pores in the hydrophobic polymer upon exposure to an aqueous liquid.

11. The dual release drug delivery system for use according to any one of the preceding embodiments, wherein the second coating further comprises hydroxypropylmethylcellulose, and/or a cyclodextrin or a derivative or polymer thereof.

12. The dual release drug delivery system for use according to any one of the preceding embodiments, wherein the second active ingredient is present in an amount equivalent to from 0.4 to 1.5 mg, for example 0.5 mg or 1.0 mg.

13. The dual release drug delivery system for use according to any one of the preceding embodiments, wherein the drug delivery system is a tablet.

14. A method for enhancing female sexual desire in the treatment of Female Sexual Interest and Arousal Disorder (FSIAD), the method comprising administering the dual release drug delivery system for use according to any one of the preceding embodiments to a subject.

15. The method according to embodiment 14, wherein the peak free testosterone level in the subject after administration is between 0.3-2%, preferably from 0.5-2%, of peak total testosterone.

16. The method according to embodiment 14 or 15, wherein the peak serum free testosterone level in the subject after administration is from 0.01 to 0.1 ng/mL, preferably from 0.025 to 0.1 ng/mL.

17. The method according to any one of embodiments 14 to 16, wherein at least 50%, preferably at least 70%, of the second active ingredient is released within 5 minutes, preferably within 3 minutes, more preferably within 1 minute, for example within 90 seconds, after administration.

18. The method according to any one of embodiments 14 to 17, wherein the administration comprises the subject holding the dual release drug delivery system under the tongue for at least 30 seconds, preferably at least 60 seconds, for example at least 90 seconds, prior to swallowing it.

19. The method according to any one of embodiments 14 to 18, wherein the plasma total concentration of the second active ingredient peaks within 1 hour, preferably within 30 minutes, after administration.

20. The method according to any one of embodiments 14 to 19, wherein the plasma concentration of the first active ingredient peaks between 3 to 6 hours, preferably between 3 to 5 hours, for example between 3 to 4 hours, after administration.

What is claimed is:

1. A method of enhancing female sexual desire in the treatment of Female Sexual Interest and Arousal Disorder (FSIAD) utilizing a dual release drug delivery system, the dual release drug delivery system comprising:
a core comprising cellulose, a filler selected from an organic and/or an inorganic salt, and a first active ingredient for delayed immediate release;
a first coating surrounding the core, the first coating comprising a hydrophobic polymer and a hydrophilic substance; and
a second coating surrounding the first coating, the second coating comprising a second active ingredient for immediate release;
wherein:
the first active ingredient comprises a PDE5 inhibitor;
the second active ingredient is a testosterone, or a functional analogue or derivative of testosterone,
the delayed release of the first active ingredient is an immediate release occurring between 2 to 6 hours after the release of the second active ingredient; and wherein:
the second active ingredient is present in an amount equivalent to from 0.3 to 1.5 mg of testosterone.

2. The method according to claim 1, wherein the PDE5 inhibitor is selected from the group consisting of sildenafil, tadalafil, vardenafil, and combinations thereof.

3. The method according to claim 1, wherein the first active ingredient is sildenafil present in an amount of from 10 to 120 mg.

4. The method according to claim 1, wherein the first active ingredient is configured for oral administration; and/or wherein the second active ingredient is configured for sublingual administration.

5. The method according to claim 1, wherein the filler is an inorganic salt and/or wherein the cellulose is microcrystalline cellulose.

6. The method according to claim 1, wherein the core further comprises a water-insoluble gel-forming disintegrant comprising cross-linked sodium carboxy methylcellulose, sodium starch glycolate and/or cross-linked polyvinylpyrrolidone.

7. The method according to claim 1, wherein the hydrophobic polymer is a hydrophobic polymeric ethyl cellulose; and/or wherein the hydrophilic substance is a water insoluble hydrophilic substance and the first coating contains pores prior to exposure to an aqueous liquid; or wherein the hydrophilic substance is a water soluble hydrophilic substance and the water soluble hydrophilic substance forms pores in the hydrophobic polymer upon exposure to an aqueous liquid.

8. The method according to claim 1, wherein the second coating further comprises hydroxypropylmethylcellulose, and/or a cyclodextrin or a derivative or polymer thereof.

9. The method according to claim 1, wherein the second active ingredient is present in an amount equivalent to from 0.4 to 1.5 mg.

10. The method according to claim 1, wherein the drug delivery system is a tablet.

11. The method according to claim 1, wherein the peak free testosterone level in the subject after administration is between 0.3-2% and/or wherein the peak serum free testosterone level in the subject after administration is from 0.01 to 0.1 ng/mL.

12. The method according to claim 11, wherein at least 50% of the second active ingredient is released within 5 minutes after administration.

13. The method according to claim 11, wherein the administration comprises the subject holding the dual release drug delivery system under the tongue for at least 30 seconds prior to swallowing it.

14. The method according to claim 11, wherein the plasma total concentration of the second active ingredient peaks within 1 hour after administration; and/or wherein the plasma concentration of the first active ingredient peaks between 3 to 6 hours after administration.

15. A pharmaceutical composition comprising testosterone or a functional analogue or derivative thereof, in combination with a PDE5 inhibitor, wherein the testosterone or functional analogue or derivative thereof is present in an amount equivalent to 1-5 mg testosterone.

16. The pharmaceutical composition of claim 16, comprising:
testosterone or functional analogue or derivative thereof present in an amount equivalent to 1 to 4 mg testosterone; and
no less than 25 mg sildenafil.

17. The pharmaceutical composition of claim 15, wherein the composition comprises:
a core comprising cellulose, a filler selected from an organic and/or an inorganic salt, and the PDE5 inhibitor for delayed immediate release;
a separating coating surrounding the core, comprising a hydrophobic polymer and a hydrophilic substance; and
an outer coating surrounding the separating coating, comprising testosterone or the functional analogue or derivative thereof, for immediate release.

18. The pharmaceutical composition of claim 15, wherein the delayed release of the PDE5 inhibitor is an immediate release occurring between 1.5 to 6 hours after the release of testosterone or the functional analogue or derivative thereof.

19. The pharmaceutical composition of claim 15, wherein the testosterone or the functional analogue or derivative thereof is configured for sublingual administration.

20. The pharmaceutical composition of claim 15, which is a tablet.

21. A method of treating a patient for Female Sexual Interest and Arousal Disorder (FSIAD), wherein the patient is post-menopausal, the patient is using combined oral contraceptives or receiving hormone replacement therapy, the patient has a BMI no greater than 22.5; and/or the patient has a plasma SHBG level of no less than 100 nmol/L, the method comprising:
administering the pharmaceutical composition of claim 15 to the patient so as to enhance female sexual desire in the patient.

22. The method according to claim 21, wherein the patient has a plasma albumin level of no greater than 37 g/L.

23. A method of treating a patient for Female Sexual Interest and Arousal Disorder (FSIAD), wherein the patient is post-menopausal, is using combined oral contraceptives (COCs), is receiving hormone replacement therapy, has a BMI (Body Mass Index) no greater than 22,5; and/or has a plasma SHBG (sex hormone-binding globulin) level of no less than 100 nmol/L, the method comprising:
administering to the patient testosterone or a functional analogue or derivative thereof in an amount equivalent to at least 1.0 mg testosterone in combination with administering a PDE5 inhibitor so as to enhance female sexual desire in the patient.

24. A method of treating a patient for Female Sexual Interest and Arousal Disorder (FSIAD), wherein the patient has a BMI (Body Mass Index) no greater than 22.5 and/or has a plasma albumin level of no greater than 37 g/L, the method comprising:
administering to the patient sildenafil in an amount of no less than 25 mg in combination with administering testosterone or a functional analogue or derivative thereof so as to enhance female sexual desire in the patient.

25. The method according to claim 24, wherein the patient is post-menopausal or the patient is using combined oral contraceptives (COCs) or receiving hormone replacement therapy; and wherein testosterone or the functional analogue or derivative thereof is used in an amount equivalent to at least 1.0 mg testosterone.

26. A method of identifying a patient suitable for administration of the pharmaceutical composition of claim 15 so as to enhance female sexual desire in the treatment of Female Sexual Interest and Arousal Disorder (FSIAD), the method comprising:
identifying whether the patient is postmenopausal, the patient's body mass index (BMI) is no greater than a pre-determined threshold, the patient is using combined oral contraceptives, and/or the patient is receiving hormone replacement therapy.

27. A method for determining a dosage for a patient in the treatment of Female Sexual Interest and Arousal Disorder (FSIAD), the method comprising:
choosing a testosterone dosage of no less than 1.0 mg in combination with a PDE5 inhibitor if:
the patient is post-menopausal,
the patient is using combined oral contraceptives or receiving hormone replacement therapy;
the patient has a BMI no greater than a pre-determined threshold, and/or
the patient has a plasma SHBG level of no less than pre-determined threshold.

28. The method according to claim 27, wherein the method further comprises choosing a dosage of no less than 75 mg for the PDE5 inhibitor.

29. A method of determining a dosage regime of a pharmaceutical composition for a patient in the treatment of Female Sexual Interest and Arousal Disorder (FSIAD), wherein the pharmaceutical composition comprises a PDE5 inhibitor and testosterone or a functional analogue or derivative; wherein the method comprises:
a) collecting information on the following impactor factors of the patient:
i) the patient's menopausal status;
ii) the patient's BMI;
iii) if the patient is using combined oral contraceptives or receiving hormone replacement therapy;
iv) the plasma SHBG level of the patient; and
v) the plasma albumin level of the patient;
b) comparing the information collected to a pre-determined schedule; and
c) choosing a dosage regime of sildenafil and/or testosterone for the patient based on the comparison.

30. A method of enhancing female sexual desire in the treatment of Female Sexual Interest and Arousal Disorder (FSIAD) of a patient, the method comprising the following steps:
a) collecting information on the following impactor factors of the patient:
i) the patient's menopausal status,
ii) the patient's BMI,
iii) whether the patient is using combined oral contraceptives or receiving hormone replacement therapy,
iv) the plasma SHBG level of the patient, and
v) the plasma albumin level of the patient;
b) comparing the information collected to a pre-determined schedule; and
c) administering a pharmaceutical composition to the patient, wherein the pharmaceutical composition comprises a PDE5 inhibitor and testosterone or a functional analogue or derivative, and wherein the dosage regime of PDE5 inhibitor and/or testosterone for the patient is determined based on the comparison of step b).

31. A dual release drug delivery system for use in enhancing female sexual desire in the treatment of Female Sexual Interest and Arousal Disorder (FSIAD), the composition comprising:
a core comprising cellulose, a filler selected from an organic and/or an inorganic salt, and delayed immediate release sildenafil in an amount of 100 mg;
a first coating surrounding the core, the first coating comprising a hydrophobic polymer and a hydrophilic substance; and
a second coating surrounding the first coating, the second coating comprising testosterone, or a functional analogue or derivative thereof in an amount equivalent to at least 1.0 mg testosterone;
wherein the delayed release of sildenafil occurs between 1.5 to 6 hours after the release of the testosterone, or the functional analogue or derivative thereof.
